(19) United States
(12) Patent Application Publication
(10) Pub. No.: US 2024/0017072 A1
(43) Pub. Date: Jan. 18, 2024

(54) Improved Neurostimulation Therapy Monitoring

(71) Applicant: Saluda Medical Pty Ltd, Macquarie Park (AU)

(72) Inventors: John Louis Parker, Macquarie Park (AU); Matthew Marlon Williams, Macquarie Park (AU); Daniel John Parker, Macquarie Park (AU); Dean Michael Karantonis, Macquarie Park (AU); Samuel Nicholas Gilbert, Macquarie Park (AU)

(73) Assignee: Saluda Medical Pty Ltd, Macquarie Park (AU)

(21) Appl. No.: 18/352,150

(22) Filed: Jul. 13, 2023

(30) Foreign Application Priority Data
Jul. 14, 2022 (AU) 2022901976
Jul. 14, 2022 (AU) 2022901977

(51) Int. Cl. A61N 1/36; A61N 1/02

(52) U.S. Cl. CPC A61N 1/36139; A61N 1/025

(57) ABSTRACT

An implantable device comprising: stimulus electrodes and measurement electrodes; a stimulus source to provide a neural stimulus to be delivered to a neural pathway of a patient to evoke a neural response; measurement circuitry configured to capture a signal window sensed on the neural pathway; and a control unit configured to implement closed-loop neurostimulation therapy by: controlling the stimulus source to provide the neural stimulus according to a stimulus intensity parameter; measuring a characteristic of the signal window; computing a feedback variable from an intensity of an evoked neural response in the signal window; adjusting the stimulus intensity parameter using the feedback variable; and repeating the controlling, measuring, computing, and adjusting to maintain the feedback variable at a target, thereby obtaining multiple measured intensities of neural responses; and computing one or more quantitative indicators of efficacy of the closed-loop neurostimulation therapy using the measured characteristics of the signal windows.

### RELATED APPLICATIONS

The current application claims priority under 35 U.S.C. 119(e) and 37 CFR 1.55 to Australian provisional patent application no. 2022901977 filed on Jul. 14, 2022 and entitled “Improved neurostimulation therapy monitoring”, and to Australian provisional patent application no. 2022901976 filed on Jul. 14, 2022 and entitled “Neurostimulation therapy program self-adaptation”. The disclosure of each of Australian provisional patent application no. 2022901977 and Australian provisional patent application no. 2022901976 is hereby incorporated herein by reference in its entirety.

### TECHNICAL FIELD

The present invention in one aspect relates to programming neurostimulation therapy and in particular the monitoring of closed-loop neurostimulation therapy programs via secondary outcomes of the therapy.

The present invention in another aspect relates to controlling a neural response to a stimulus, and in particular to the adjustment of therapy parameters in response to patient reported outcomes.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation system applies an electrical pulse (stimulus) to neural tissue (fibres, or neurons) in order to generate a therapeutic effect. In general, the electrical stimulus generated by a neuromodulation system evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a system typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. The fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ (A-beta) fibres. When recruitment is too large, A-beta fibres produce uncomfortable sensations. Stimulation at high intensity may even recruit Aδ (A-delta) fibres, which are sensory nerve fibres associated with acute pain, cold and pressure sensation. It is therefore desirable to maintain stimulus intensity within a therapeutic range between the recruitment threshold and the discomfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Another control problem facing neuromodulation systems of all types is achieving neural recruitment at a sufficient level for therapeutic effect, but at minimal expenditure of energy. The power consumption of the stimulation paradigm has a direct effect on battery requirements which in turn affects the device's physical size and lifetime. For rechargeable systems, increased power consumption results in more frequent charging and, given that batteries only permit a limited number of charging cycles, ultimately this reduces the implanted lifetime of the device.

Attempts have been made to address such problems by way of feedback or closed-loop control, such as using the methods set forth in International Patent Publication No. WO2012/155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately detect and record a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

However, neural response measurement can be a difficult task as a neural response component in the sensed signal will typically have a maximum amplitude in the range of microvolts. In contrast, a stimulus applied to evoke the response is typically several volts, and manifests in the sensed signal as crosstalk of that magnitude. Moreover, stimulus generally results in electrode artefact, which manifests in the sensed signal as a decaying output of the order of several millivolts after the end of the stimulus. As the neural response can be contemporaneous with the stimulus crosstalk and/or the stimulus artefact, neural response measurements present a difficult challenge of measurement amplifier design. For example, to resolve a 10 μV ECAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result a time-decaying artefact waveform of positive or negative polarity, their identification and elimination can be laborious.

Closed-loop neurostimulation therapy is governed by a number of parameters to which values must be assigned to implement the therapy. The effectiveness of the therapy depends in large measure on the suitability of the assigned parameter values to the patient undergoing the therapy. As patients vary significantly in their physiological characteristics, a “one-size-fits-all” approach to parameter value assignment is likely to result in ineffective therapy for a large proportion of patients. An important preliminary task, once a neurostimulation therapy device has been implanted in a patient, is therefore to assign values to the therapy parameters that maximise the effectiveness of the therapy the device will deliver to that particular patient. This task is known as programming or fitting the device.

Programming generally involves applying certain test stimuli via the device, recording responses, and based on the recorded responses, inferring or calculating the most effective therapy parameter values for the patient. The resulting therapy parameter values are then formed into a “program” that may be loaded to the device to govern subsequent therapy.

Some of the recorded responses may be neural responses evoked by the test stimuli, which provide an objective source of information that may be analysed along with subjective responses elicited from the patient. In an effective programming system, the more responses that are analysed, the more effective the eventual assigned therapy parameter values should be. Other responses are obtained from patient reporting of their sensations. In one example, patients are asked to represent their pain sensations on a visual analogue scale (VAS).

However, programming may be costly and time-consuming if unnecessarily prolonged. There is therefore an incentive to minimise the number of test stimuli to be applied and the amount of information to be recorded and analysed in order to produce the assigned values of the therapy parameters that make up a therapy program.

In addition, circumstances may change over time as the patient receives therapy, such that a program that was initially appropriate is no longer appropriate to treat the patient's pain. Examples of changes in circumstances include lead migration within the spinal column, changes in medication, and changes in neuropathology. It is therefore desirable for a neurostimulation system to continue to obtain and analyse responses to therapy so as to monitor the efficacy of its therapy. If the therapy loses enough efficacy as determined through such self-monitoring, a flag may be raised and action may be taken to re-program the patient to ensure its continuing efficacy.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are systems and methods configured to monitor the efficacy of implantable closed-loop neurostimulation therapy for chronic pain by analysing neural responses measured by the neuromodulation device. Such analysis may produce quantitative indications of therapy efficacy via secondary outcomes of the closed-loop neurostimulation therapy. Secondary outcomes are to be contrasted with primary outcomes of closed-loop neurostimulation therapy for chronic pain, which are those directly related to pain relief, such as pain scores. The quantitative indications of therapy efficacy obtained from secondary outcomes may be monitored and if they travel beyond predetermined limits of acceptable efficacy, an indication may be transmitted that re-programming would be worthwhile. In addition, or alternatively, the quantitative indications may be used to adapt the program automatically, whereby changes that improve the efficacy as measured by the quantitative indications are encouraged, and those that do not are deprecated.

According to a first aspect of the present technology, there is provided an implantable device for controllably delivering a neural stimulus. The device comprises: a plurality of electrodes including one or more stimulus electrodes and one or more measurement electrodes; a stimulus source configured to provide neural stimuli to be delivered via the one or more stimulus electrodes to a neural pathway of a patient in order to evoke a neural response on the neural pathway; measurement circuitry configured to capture signal windows sensed on the neural pathway via one or more measurement electrodes subsequent to the respective neural stimuli; and a control unit. The control unit is configured to implement closed-loop neurostimulation therapy by: controlling the stimulus source to provide a neural stimulus according to a stimulus intensity parameter; measuring a characteristic of the signal window; computing a feedback variable from an intensity of an evoked neural response in the signal window; adjusting the stimulus intensity parameter using the feedback variable; and repeating the controlling, measuring, computing, and adjusting so as to maintain the feedback variable at a target, thereby obtaining multiple measured intensities of neural responses. The control unit is further configured to compute one or more quantitative indicators of efficacy of the closed-loop neurostimulation therapy using the measured characteristics of the signal windows.

According to a second aspect of the present technology, there is provided an automated method of controllably delivering a neural stimulus. The method comprises: controlling a stimulus source to provide a neural stimulus to be delivered, via one or more stimulus electrodes, to a neural pathway of a patient in order to evoke a neural response on the neural pathway, the neural stimulus being delivered according to a stimulus intensity parameter; capturing a signal window sensed on the neural pathway, via one or more measurement electrodes, subsequent to the neural stimulus; measuring a characteristic of the signal window; computing a feedback variable, from an intensity of an evoked neural response in the signal window; adjusting the stimulus intensity parameter using the feedback variable; and repeating the controlling, measuring, computing, and adjusting so as to maintain the feedback variable at a target, thereby obtaining multiple measured intensities of neural responses. The control unit is configured to compute one or more quantitative indicators of efficacy of the closed-loop neurostimulation therapy using the measured characteristics of the signal window.

Also disclosed herein is a patient-responsive method and system for programming a neuromodulation device, wherein the programming comprises determining a preferred therapy program based on patient reported outcomes.

According to an aspect of the present technology, there is provided a method for controllably generating neural stimuli for delivery to a patient. The method comprises controlling a stimulus source to generate neural stimuli according to a first stimulus program and receiving, via a communication interface, a patient reported outcome. The method further comprises, in response to receiving the patient reported outcome, determining a preferred stimulus program, and controlling the stimulus source to generate neural stimuli according to the preferred stimulus program.

In one embodiment, the patient reported outcome comprises a first patient reported outcome. In one embodiment, determining a preferred stimulus program comprises controlling the stimulus source to generate the neural stimuli according to a second stimulus program, receiving, via the communication interface, a second patient reported outcome, and in response to receiving the second patient reported outcome, determining a preferred stimulus program based on the first patient reported outcome and the second patient reported outcome.

In one embodiment, the first stimulus program comprises one or more therapy parameters, wherein the therapy parameters comprise at least one of: a pulse width; a pulse type; a stimulus intensity; a stimulus frequency; a stimulus electrode configuration; a measurement electrode configuration; or a target ECAP amplitude.

In one embodiment, the first stimulus program differs from the preferred stimulus program in terms of one or more of the therapy parameters. In one embodiment, the patient reported outcome comprises a quantitative indication of patient satisfaction with the first stimulus program.

In one embodiment, determining the preferred stimulus program comprises adjusting at least one of the one or more parameters of the first stimulus program. In one embodiment, determining the preferred stimulus program comprises selecting the preferred stimulus program from a set of candidate stimulus programs. In one embodiment, the set of candidate stimulus programs comprises the first stimulus program and a second stimulus program.

In one embodiment, determining the preferred stimulus program comprises determining an unsatisfactory stimulus program from the set of candidate stimulus programs, and, in response to determining the unsatisfactory stimulus program, adjusting the set of candidate stimulus programs. In one embodiment, determining an unsatisfactory stimulus program comprises receiving an adverse patient reported outcome.

In one embodiment, adjusting the set of candidate stimulus programs comprises removing the unsatisfactory stimulus program from the set of candidate stimulus programs.

In one embodiment, determining a preferred stimulus program comprises determining a first time period associated with generating the neural stimuli according to the first stimulus program, and determining the preferred stimulus program based on the first time period.

In one embodiment, determining a preferred stimulus program further comprises controlling the stimulus source to generate the neural stimuli according to a second stimulus program, receiving, via the communication interface, a second patient reported outcome, in response to receiving the second patient reported outcome, determining a second time period associated with generating the neural stimuli according to the second stimulus program, and determining the preferred stimulus program based on the first time period and the second time period.

In one embodiment, controlling the stimulus source to generate the neural stimuli according to a first stimulus program comprises controlling the stimulus source to generate the neural stimuli according to the first stimulus program for a first set time period, and receiving a first set of patient reported outcomes during the first set time period. In one embodiment, determining a preferred stimulus program comprises controlling the stimulus source to generate the neural stimuli according to a second stimulus program for a second set time period, receiving a second set of patient reported outcomes during the second set time period, and determining a preferred stimulus program based on the first set of patient reported outcomes and the second set of patient reported outcomes.

In one embodiment, the method further comprises transmitting a prompt signal to a patient control interface, wherein the prompt signal is configured to prompt the patient to provide a patient reported outcome. In one embodiment, determining the preferred stimulus program comprises determining a power consumption level associated with the preferred stimulus program. In one embodiment, determining the preferred stimulus program comprises determining a measurement of compliance associated with the preferred stimulus program.

In one embodiment, the first stimulus program is associated with a first set of candidate stimulus programs. In one embodiment, determining a preferred stimulus program comprises determining a second set of candidate stimulus programs based on the patient reported outcome and the first stimulus program, and selecting the preferred stimulus program from the second set of candidate stimulus programs.

In one embodiment, each stimulus program of the first set of candidate stimulus programs comprises a first therapy parameter set to a different parameter value, and each stimulus program of the second set of candidate stimulus programs comprises the first therapy parameter set to a different parameter value that is based on the parameter value of the therapy parameter of the first stimulus program.

In one embodiment, determining the second set of candidate stimulus programs comprises applying Bayesian analysis to the patient reported outcome and the first set of candidate stimulus programs.

According to another aspect of the present technology, there is provided an implantable device for controllably generating neural stimuli for delivery to a patient. The device comprises a stimulus source configured to generate neural stimuli to be delivered to a neural pathway of the patient in order to evoke a neural response on the neural pathway, and a control unit. The control unit is configured to control a stimulus source to generate the neural stimuli according to a first stimulus program, and receive, via a communication interface, a patient reported outcome. The control unit is further configured to, in response to receiving the patient reported outcome, determine a preferred stimulus program, and control the stimulus source to generate neural stimuli according to the preferred stimulus program.

In one embodiment, the device further comprises the communication interface configured to receive the patient reported outcome. In one embodiment, the communication interface is further configured to transmit a prompt signal to a patient control interface.

In one embodiment, the patient reported outcome comprises a first patient reported outcome, and the determining a preferred stimulus program comprises: controlling the stimulus source to generate the neural stimuli according to a second stimulus program; receiving, via the communication interface, a second patient reported outcome; and in response to receiving the second patient reported outcome, determining a preferred stimulus program based on the first patient reported outcome and the second patient reported outcome.

In one embodiment, the first stimulus program comprises one or more therapy parameters, and the therapy parameters comprise at least one of: a pulse width; a pulse type; a stimulus intensity; a stimulus frequency; a stimulus electrode configuration; a measurement electrode configuration; or a target ECAP amplitude.

In one embodiment, the first stimulus program differs from the preferred stimulus program in terms of one or more of the therapy parameters.

In one embodiment, determining the preferred stimulus program comprises adjusting at least one of the one or more parameters of the first stimulus program.

In one embodiment, determining the preferred stimulus program comprises selecting the preferred stimulus program from a set of candidate stimulus programs.

According to another aspect of the present technology, there is provided a neurostimulation system. The neurostimulation system comprises an implantable device for controllably generating neural stimuli for delivery to a patient. The implantable device comprises a stimulus source configured to generate neural stimuli to be delivered to a neural pathway of the patient in order to evoke a neural response on the neural pathway, and a control unit. The control unit is configured to control a stimulus source to generate the neural stimuli according to a first stimulus program, and receive, via a communication interface, a patient reported outcome. The control unit is further configured to, in response to receiving the patient reported outcome, determine a preferred stimulus program, and control the stimulus source to generate neural stimuli according to the preferred stimulus program. The neurostimulation system further comprises an external computing device in communication with the communication interface of the implantable device. The external computing device comprises a patient control interface, and a processor. The processor is configured to receive, via the patient control interface, the patient reported outcome, and transmit the patient reported outcome to the communication interface.

In one embodiment, the processor is further configured to receive a prompt signal from the communication interface, and render, on the patient control interface, a prompt for a patient reported outcome.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1, according to one implementation of the present technology;

FIG. 3 is a schematic illustrating interaction of the implanted stimulator of FIG. 1 with a nerve, according to one implementation of the present technology;

FIG. 4a illustrates an idealised activation plot for one posture of a patient undergoing neurostimulation;

FIG. 4b illustrates the variation in the activation plots with changing posture of the patient;

FIG. 5 is a schematic illustrating elements and inputs of a closed-loop neural stimulation system, according to one implementation of the present technology;

FIG. 6 illustrates the typical form of an electrically evoked compound action potential (ECAP) of a healthy subject;

FIG. 7 is a block diagram of a neuromodulation therapy system including the implanted stimulator of FIG. 1 according to one implementation of the present technology;

FIG. 8a is a flow chart illustrating a method of monitoring the efficacy of implantable closed-loop neural stimulation (CLNS) therapy for chronic pain by analysing neural responses measured by an implantable neuromodulation device, according to one aspect of the present technology;

FIG. 8b is a flow chart illustrating a method of monitoring the efficacy of implantable CLNS therapy for chronic pain by analysing neural responses measured by an implantable neuromodulation device, according to a further aspect of the present technology;

FIG. 9 is a block diagram illustrating the data flow of a neuromodulation therapy system, according to one implementation of the present technology;

FIG. 10 illustrates a patient control interface, according to one implementation of the present technology;

FIG. 11 illustrates a method, performed by the control unit, to determine a preferred stimset based on a patient reported outcome, according to one implementation of the present technology;

FIGS. 12a and 12b illustrate a tree-structured set of candidate stimsets, according to one implementation of the present technology;

FIG. 13 illustrates a scenario in which the control unit applies a time-based method to determine a preferred stimset from a plurality of sets of candidate stimsets, according to one implementation of the present technology;

FIG. 14 illustrates an example in which the control unit is configured to determine a preferred stimset based on a consideration of the number of adverse PROs that are received by the control unit, according to one implementation of the present technology;

FIG. 15 illustrates an example scenario in which the control unit utilises prompts to obtain patient reported outcomes, according to one implementation of the present technology;

FIG. 16 illustrates an example scenario in which the control unit switches from applying a default stimset to applying a candidate stimset, according to one implementation of the present technology;

FIG. 17 is a flow chart illustrating a method of determining new set of candidate stimsets from a set of candidate stimsets, according to one implementation of the present technology; and

FIGS. 18a and 18b illustrate a tree-structured set of candidate stimsets, according to one implementation of the present technology.

### DETAILED DESCRIPTION OF THE PRESENT TECHNOLOGY

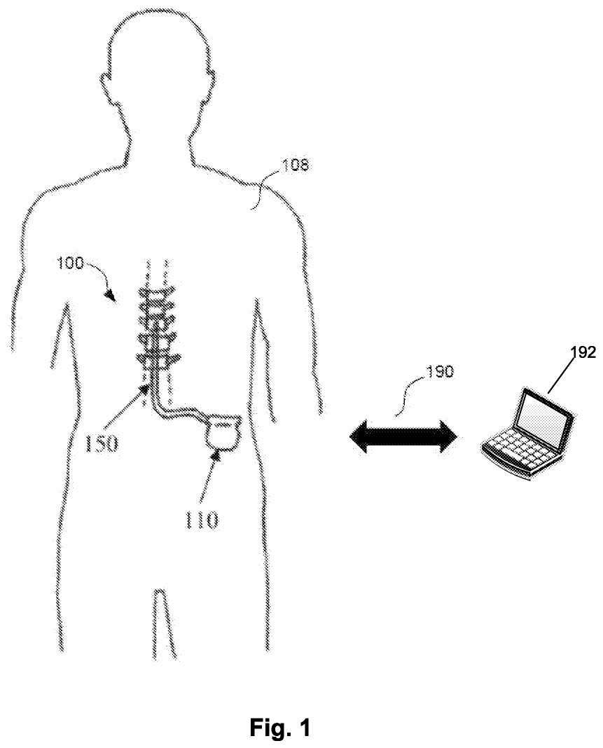

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as a flank or sub-clavicular. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

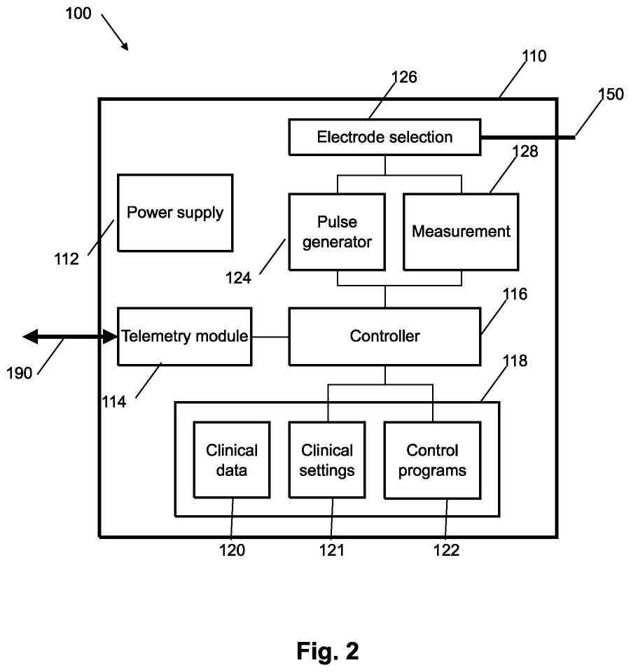

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and inductive transfer, may be used by telemetry module 114 to transfer power and/or data to and from the electronics module 110 via communications channel 190. Module controller 116 has an associated memory 118 storing one or more of clinical data 120, clinical settings 121, control programs 122, and the like. Controller 116 is configured by the control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

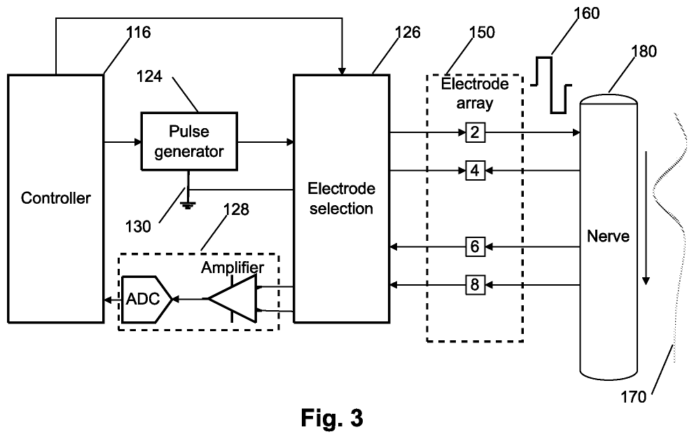

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus charge recovery in each phase, to maintain a zero net charge transfer. Because a given electrode may act as both a stimulus and a return electrode over a complete multiphasic stimulus pulse, both electrodes are generally referred to as stimulus electrodes. The use of two electrodes in this manner for delivering and recovering current in each stimulus phase is referred to as bipolar stimulation. Alternative embodiments may apply other forms of bipolar stimulation, or may use a greater number of stimulus electrodes. The set of stimulus electrodes and their respective polarities is referred to as the stimulus electrode configuration. Electrode selection module 126 is illustrated as connecting to a ground 130 of the pulse generator 124 to enable stimulus charge recovery via the return electrode 4. However, other connections for charge recovery may be used in other implementations.

Delivery of an appropriate stimulus from stimulus electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the stimulus electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus configuration is found which evokes paraesthesia in a location and of a size which is congruent with the area of the patient's body affected by pain and of a quality that is comfortable for the patient, the clinician or the patient nominates that configuration for ongoing use. The program parameters may be loaded into the memory 118 of the stimulator 100 as the clinical settings 121.

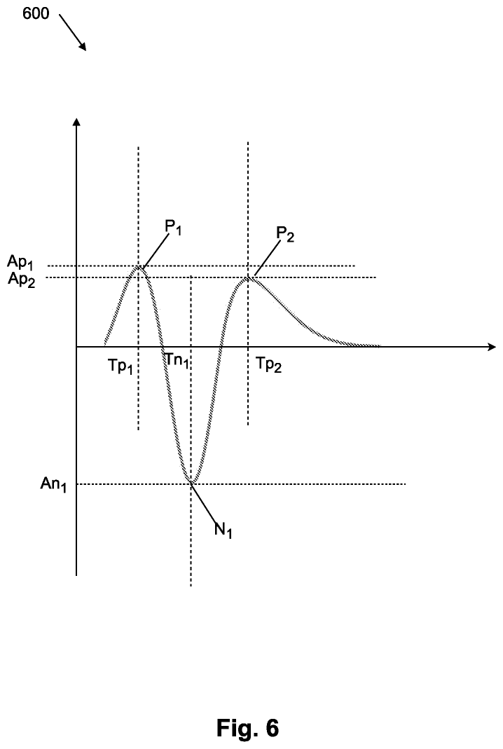

FIG. 6 illustrates the typical form of an ECAP 600 of a healthy subject, as recorded at a single measurement electrode referenced to the system ground 130. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be parametrised by any suitable parameter(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to detect the existence and measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented by controller 116, configured by control programs 122, to obtain information regarding the effect of the applied stimulus upon the nerve 180. In some implementations, the sensed signals are processed by the ECAP detector in a manner which extracts and stores one or more parameters from each evoked neural response or group of evoked neural responses contained in the sensed signal. In one such implementation, the parameter comprises a peak-to-peak ECAP amplitude in microvolts (μV). For example, the neural responses may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may extract and store an alternative parameter from the neural response, or may extract and store two or more parameters from the neural response.

Stimulator 100 applies stimuli over a potentially long period such as days, weeks, or months and during this time may store parameters of neural responses, clinical settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more parameters such as a measure of the amplitude of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

An activation plot, or growth curve, is an approximation to the relationship between stimulus intensity (e.g. an amplitude of the current pulse 160) and intensity of neural response 170 evoked by the stimulus (e.g. an ECAP amplitude).

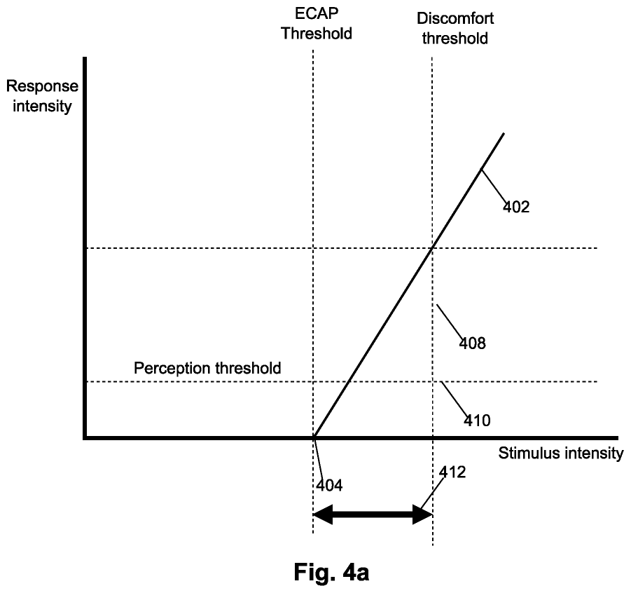

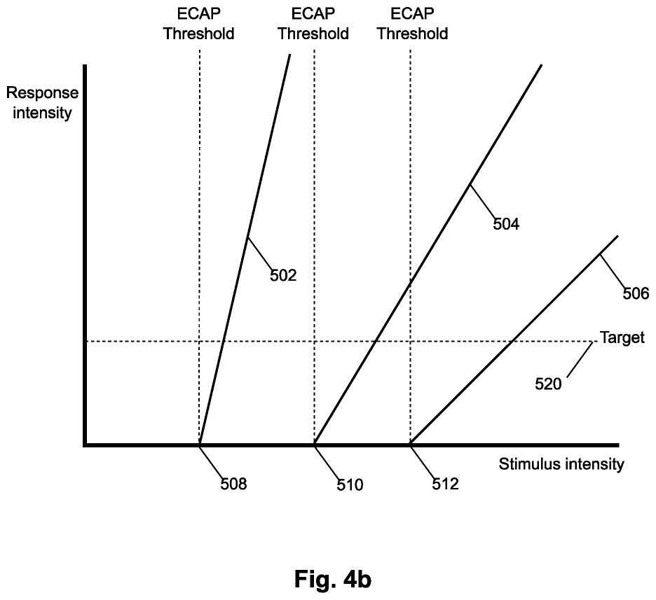

FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s - T ) , & s \geq T \\ 0 , & s < T\end{matrix} \qquad ( 1 )$$

where s is the stimulus intensity, y is the ECAP amplitude, T is the ECAP threshold and S is the slope of the activation plot (referred to herein as the patient sensitivity). The slope S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is perceivable by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more extracted ECAP parameters. In one implementation, the device may adjust the stimulus intensity to maintain the extracted ECAP amplitude at a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on an extracted ECAP parameter is said to be operating in closed-loop mode and will also be referred to as a closed-loop neural stimulation (CLNS) device. By adjusting the applied stimulus intensity to maintain the extracted ECAP amplitude at an appropriate target response intensity, such as an ECAP target 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is characterised by multiple parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The evoked neural response (e.g. an ECAP) is detected, and its amplitude measured by the CLNS device and compared to the target response intensity.

The measured neural response amplitude, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

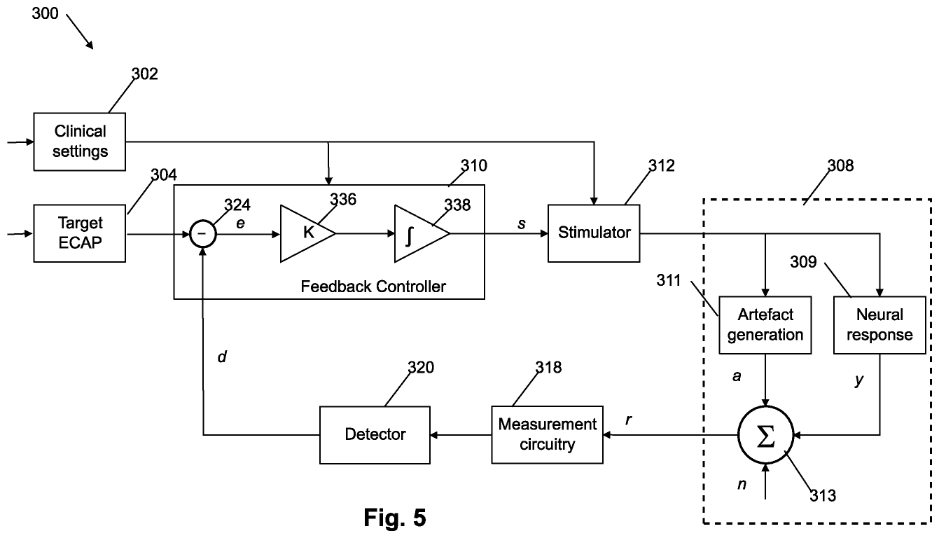

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and noise), and samples the amplified sensed signal r to capture a “signal window” comprising a predetermined number of samples of the amplified sensed signal r. The ECAP detector 320 processes the signal window and outputs a measured neural response intensity d. In one implementation, the neural response intensity comprises a peak-to-peak ECAP amplitude. The measured response intensity d is input into the feedback controller 310.

The feedback controller 310 comprises a comparator 324 that compares the measured response intensity d to the target ECAP amplitude as set by the target ECAP controller 304. In one embodiment, the target ECAP controller 304 is part of the control unit 116. In one embodiment, the control unit 116 sets the target ECAP amplitude (otherwise referred to as the target ECAP). In some embodiments, the control unit 116 is configured to receive input, from the patient 108 or the external computing device 192 via the communication interface 114, regarding the target ECAP amplitude or indicating a request to increase or decrease the target ECAP amplitude.

In one embodiment, the comparator 324 provides an indication of the difference between the measured response intensity d and the target ECAP amplitude. This difference is the error value, e. The error value e is input into the feedback controller 310.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be computed by the feedback controller 310 as

s=∫Kedt (2)

where K is the gain of the gain element 336 (the controller gain).

This relation may also be represented as

δs=Ke

where δs is an adjustment to the current stimulus intensity parameter s.

A target ECAP amplitude is input to the feedback controller 310 via the target ECAP controller 304. In one embodiment, the target ECAP controller 304 provides an indication of a specific target ECAP amplitude. In another embodiment, the target ECAP controller 304 provides an indication to increase or to decrease the present target ECAP amplitude. The target ECAP controller 304 may comprise an input into the CLNS system 300, via which the patient or clinician can input a target ECAP amplitude, or indication thereof. The target ECAP controller 304 may comprise memory in which the target ECAP amplitude is stored, and provided to the feedback controller 310.

A clinical settings controller 302 provides clinical settings to the system 300, including the feedback controller 310 and the stimulus parameters for the stimulator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 10 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

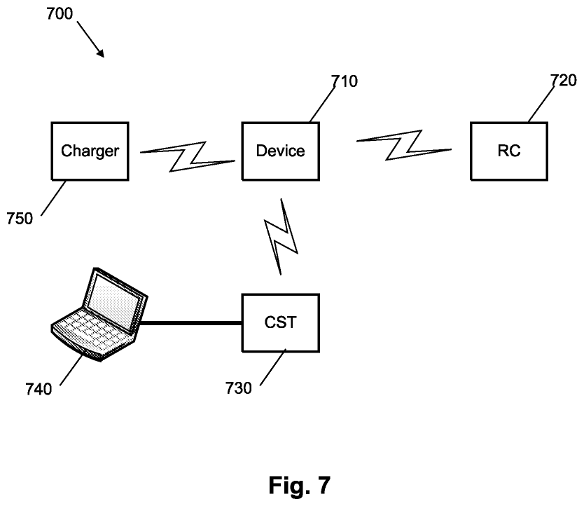

FIG. 7 is a block diagram of a neuromodulation system 700. The neuromodulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target neural response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### Closed-Loop Neurostimulation Therapy Monitoring

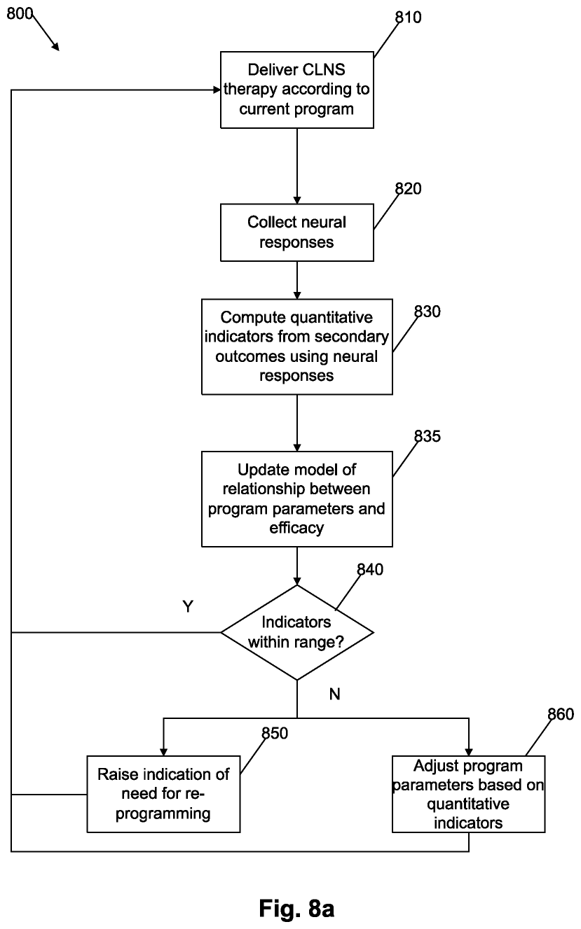

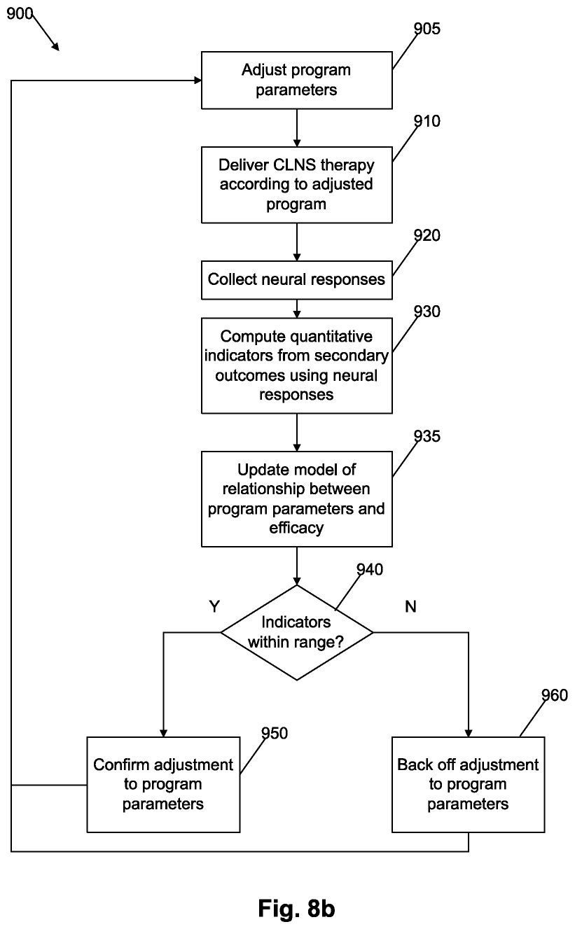

FIG. 8a is a flow chart illustrating a method 800 of monitoring the efficacy of implantable closed-loop neural stimulation (CLNS) therapy for chronic pain by analysing neural responses measured by an implantable neuromodulation device such as the stimulator 100 of FIG. 2 or the neuromodulation device 710 of FIG. 7, according to one aspect of the present technology. The method 800 may be carried out by the controller of the neuromodulation device, e.g. the controller 116 of the stimulator 100, which may be configured to carry out the method 800 by the control programs 122.

The method 800 starts at step 810, which delivers CLNS therapy in accordance with the current program stored in the memory of the neuromodulation device as described above. At the first iteration of step 810, the current program is an initial program that has been fitted to the patient and the device. The initial program may be a default program, chosen for the patient on the basis of demographic and other patient information, or a personalised program derived from a fitting session.

Step 820 collects and analyses signal windows captured via the measurement electrode configuration. The signal windows collected at step 820 may contain evoked neural responses such as ECAPs. The signal windows collected at step 820 may also, or alternatively, contain non-evoked neural responses measured in between stimulus pulses when no ECAPs are present, or during “off” periods when no stimulation is taking place. International Patent Publication no. WO2016077882 by the present applicant, the contents of which are herein incorporated by reference, describes the discrimination of non-evoked neural responses from evoked neural responses to stimuli.

Step 830 then computes one or more quantitative indicators (QIs) of therapy efficacy using one or more characteristics of the neural responses in the collected signal windows. The QIs are computed from secondary outcomes of the CLNS therapy as indicated by the measured neural responses. Examples of the computation carried out at step 830 are described below. Optionally, for greater confidence, step 830 may also use data from supplementary specialised sensors either embedded in, or in communication with, the neuromodulation device. Such data may comprise blood pressure, EEG, heart rate variability, activity, voice characteristics, facial imaging, and pupil dilation reflex.

At the next step 835, the controller updates an internal model of the relationship between the current program parameters (clinical settings) and related device performance (neural responses), and the efficacy of the therapy based on the secondary outcome QIs computed at step 830. Step 835 may use embedded machine learning or recursive decision flow to update the model.

Step 840 then compares the computed one or more QIs from step 830 with respective predetermined ranges indicating normal, expected efficacy. If all the QIs are within the normal ranges (“Y”), the method 800 returns to step 810. Otherwise (“N”), either of step 850 and 860 may be executed. Step 850 transmits an indicator indicating a need for re-programming. The indicator may be transmitted by the device to an external computing device such as the patient remote control 720 and thereby, via a visual or audio indicator on the remote control 720, to a user of the external computing device. Alternatively or in addition, step 840 may comprise comparing the computed one or more QIs from step 830 with the QIs computed from a previous iteration of the method 800 (e.g. the immediately preceding iteration, or a number of previous iterations), and checking whether a preceding adjustment in the program parameters lead to an improvement in one or more of the computed QIs. When an improvement is recorded, then the method can return to step 810; and when a decline in the computed QIs is recorded, either of step 850 or 860 may be executed. This comparing and checking of QIs computed from previous iterations may be performed even where the computed one or more QIs from step 830 fall outside of their respective predetermined ranges. In certain embodiments, therefore, recording of improvements in the one or more computed QIs can iteratively move the device towards giving the patient optimal stimulation, even if QI values are outside of a normal range.

Step 860 adjusts the program parameters in accordance with the QIs, in particular the out-of-range QI, and the model updated at step 835, with the aim of improving the therapy efficacy.

After either step 850 or step 860, the method 800 returns to step 810 to continue therapy with either the original or the adjusted program.

Over many iterations of step 860 of the method 800, the device slowly learns how to give the patient the optimal stimulation, because the device can objectively measure secondary outcomes via neural responses, without having to rely on patient-reported primary outcomes.

FIG. 8b is a flow chart illustrating a method 900 of monitoring the efficacy of implantable closed-loop neurostimulation (CLNS) therapy for chronic pain by analysing neural responses measured by an implantable neuromodulation device such as the stimulator 100 of FIG. 2 or the neuromodulation device 710 of FIG. 7, according to a further aspect of the present technology. The method 900 may be carried out by the controller of the neuromodulation device, e.g. the controller 116 of the stimulator 100, which may be configured to carry out the method 800 by the control programs 122.

The method 900 starts at step 905, which adjusts one or more parameters of the current program stored in the memory of the neuromodulation device as described above. At the first iteration of step 905, the current program is an initial program that has been fitted to the patient and the device. The initial program may be a default program, chosen for the patient on the basis of demographic and other patient information, or a personalised program derived from a fitting session.

Step 910 then delivers CLNS therapy in accordance with the adjusted program, as in step 810 of the method 800.

Step 920 collects and stores signal windows captured via the measurement electrode configuration, as in step 820 of the method 800.

Step 930 then computes one or more quantitative indicators (QIs) of therapy efficacy using one or more characteristics of the neural responses in the collected signal windows. The QIs are computed from secondary outcomes of the CLNS therapy as indicated by the measured neural responses, as at step 830 of the method 800.

At the next step 935, the controller updates an internal model of the relationship between the current program parameters (clinical settings) and related device performance (neural responses), and the efficacy of the therapy based on the secondary outcome QIs computed at step 930, as at step 835 of the method 800.

Step 940 then compares the computed one or more QIs from step 930 with respective predetermined ranges indicating normal, expected efficacy. If all the QIs are within the normal ranges (“Y”), step 950 confirms the adjustment to the program parameters made at step 905. If not (“N”), step 960 backs off, or cancels, the adjustment to the program parameters made at step 905. After either step 950 or step 960, the method 900 returns to step 905 to make another adjustment to the program parameters. Over many iterations of step 950 or step 960 of the method 900, the device slowly learns how to give the patient the optimal stimulation, because the device can objectively measure secondary outcomes via neural responses, without having to rely on patient-reported primary outcomes. Alternatively or in addition, step 940 may comprise comparing the computed one or more QIs from step 930 with the QIs computed from a previous iteration of the method (e.g. the immediately preceding iteration, or some number of previous iterations), and checking whether an adjustment in the program parameters leads to an improvement or a decline in one or more of the computed QIs. When there is an improvement, then step 950 can confirm the adjusted parameters; and when there is a decline in the computed QIs, step 960 can back off or cancel the adjustment. Comparing and checking of QIs computed from previous iterations may be performed even where the computed one or more QIs from step 930 fall outside of their respective predetermined ranges. It can therefore be understood that recorded QI improvements, even if they are out of a normal range, may iteratively move the device towards giving the patient optimal stimulation.

One example of objectively measuring a secondary outcome using neural response data, as may be used at step 830 of the method 800 or step 930 of the method 900, is to use evoked neural response intensity (e.g. ECAP amplitude) along with the intensity of the corresponding stimuli to estimate posture. International Patent Publication no. WO2022040757, the contents of which are herein incorporated by reference, describes how to estimate posture at selected time intervals from neural response intensity and stimulus intensity data collected over the selected time intervals. In one example, during programming, two-dimensional histograms of ECAP amplitude and stimulus current are captured as the patient moves between various postures. Then during therapy, a two-dimensional histogram may be captured over an interval and compared with the histograms captured during programming to obtain an estimate of posture during the interval. In another example described in the above-mentioned WO2022040757, a posture relative to a reference posture may be estimated by dividing a quantity called the “refcap” by the measured ECAP amplitude. The refcap is the equivalent ECAP amplitude that would have been observed in the reference posture in response to the same stimulus that evoked the measured ECAP.

Once a measure of posture has been estimated, a measure of sleep quality may be computed by analysing changes of posture during the night-time hours. The resulting sleep quality measure is an example of a quantitative indicator of therapy efficacy based on a secondary outcome of CLNS pain relief, since sleep quality is correlated with the patient's wellbeing.

Another example of objectively measuring a secondary outcome using neural response data, as may be used at step 830 of the method 800 or step 930 of the method 900, is the analysis of non-evoked neural activity to determine the amount of rapid eye movement (REM) sleep. During REM sleep, the brain paralyses the muscles so dreaming does not cause inadvertent movement, risking injury. Therefore, motor neuron activity is measurably reduced. REM sleep may therefore be detectable through a sustained decrease in non-evoked neural activity. A quantitative indicator of sleep quality may then be inferred from the number of detected REM sleep intervals, their duration, and their consistency from night to night.

Another example of objectively measuring a secondary outcome using neural response data, as may be used at step 830 of the method 800 or step 930 of the method 900, is to use ECAP-based comfort/wellbeing estimation. Change in the ECAP amplitude over time (even pulse-to-pulse) may be used as a metric of comfort or subjective sensation strength. In one such example, a variation in synchrony with heartbeat is detectable in the ECAP amplitude or the stimulus intensity, particularly in cervical patients. From the frequency of this variation, the heart rate and its variability may be computed. The amount of variability in heart rate, independent of absolute magnitude, may be indicative of a patient's wellbeing. For example, less heart rate variability is correlated with less activity, less wellbeing, and therefore greater discomfort.

One example of adjusting the program parameters, as may be employed at step 905 of the method 900, is suitable for a program comprising multiple interleaved stimulation sets (“stimsets”) as described in International Patent Application no. PCT/AU2023/050481, the contents of which are herein incorporated by reference. A stimset is a stimulus electrode configuration (SEC), along with the stimulus parameters that govern the stimulation pulses delivered through that SEC. The stimulation pulses delivered from each stimset are interleaved with each other in a repeating cycle. In this example, the program adjustment is to randomly remove one of the stimsets. If the quantitative indicators do not deteriorate, indicating no loss of efficacy, after the stimset is removed, the removed stimset may be deemed “surplus” and its removal confirmed at step 950. Otherwise, the removed stimset is restored to the program at step 960. Over time, the method 900 will pare the multiple stimsets down to the minimum efficacious set of stimsets.

Another example of adjusting the program parameters, as may be employed at step 905 of the method 900, is to decrease the target ECAP amplitude gradually over a night. If the quantitative indicators indicate a loss of efficacy at step 940, the target ECAP amplitude may be restored to its original value at step 960 and then decreased during the next night at a lower rate. The method 900 will thereby eventually converge on the minimum effective target ECAP amplitude.

Power consumption could also be taken into account along with the secondary outcomes when computing the quantitative indicators at steps 830 and 930. For example, the quantitative indicators could be increased for programs, stimsets, or combinations of parameters that consume less power. In such implementations, the efficacy of programs, stimsets, or combinations of parameters is balanced with the consumption of power when converging on the optimal stimulation.

Measures of compliance (e.g. how much a patient is using a device) may also be taken into account when computing the quantitative indicators at steps 830 and 930. For example, the quantitative indicators could be increased for programs, stimsets, or combinations of parameters that encourage patient compliance. In such implementations, the efficacy of programs, stimsets, or combinations of parameters can factor in the level of patient compliance when converging on the optimal stimulation.

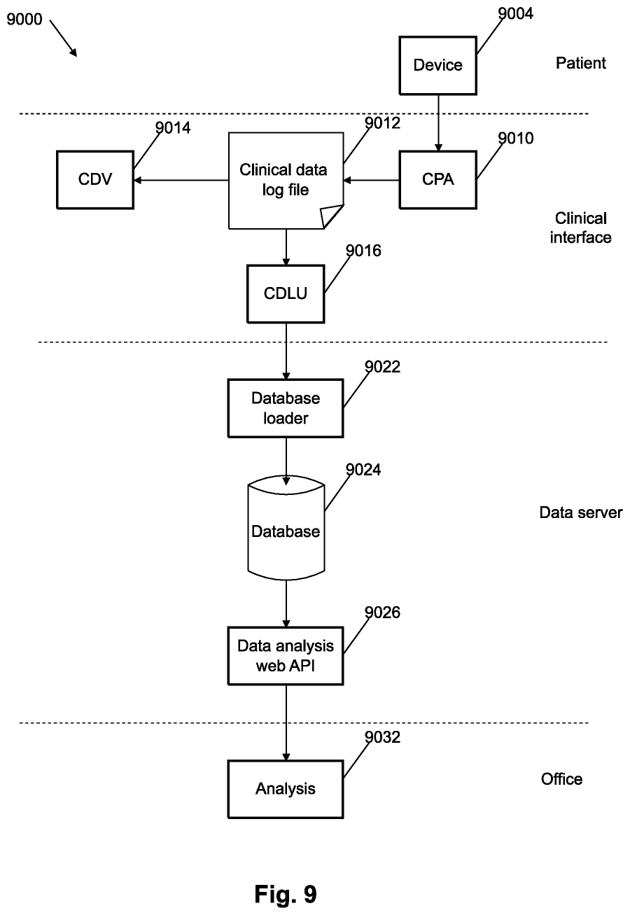

FIG. 9 is a block diagram illustrating the data flow 9000 of a neuromodulation therapy system such as the system 700 of FIG. 7 according to one implementation of the present technology. Neuromodulation device 9004, once implanted within a patient, applies stimuli over a potentially long period such as weeks or months and records neural responses, clinical settings, paraesthesia target level, and other operational parameters, discussed further below. Neuromodulation device 9004 may comprise a Closed-Loop Stimulator (CLS), in that the recorded neural responses are used in a feedback arrangement to control clinical settings on a continuous or ongoing basis. To effect suitable SCS therapy, neuromodulation device 9004 may deliver tens, hundreds or even thousands of stimuli per second, for many hours each day. The feedback loop may operate for most or all of this time, by obtaining neural response recordings following every stimulus, or at least obtaining such recordings regularly. Each recording generates a feedback variable such as a measure of the amplitude of the evoked neural response, which in turn results in the feedback loop changing at least one therapy parameter for a following stimulus. Neuromodulation device 9004 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 9004 transmits data, e.g. via communications interface 114, to a clinical programming application (CPA) 9010 installed on a clinical interface. In one implementation, the clinical interface is the CI 740 of FIG. 7. The data can be grouped into two main sources: (1) Data collected in real-time during a programming session; (2) Data downloaded from a stimulator after a period of non-clinical use by a patient. CPA 911 collects and compiles the data into a clinical data log file 9012.

All clinical data transmitted by the neuromodulation device 9004 may be compressed by use of a suitable data compression technique before transmission by communications interface 114 and/or before storage into the memory 118 to enable storage by neuromodulation device 9004 of higher resolution data. This higher resolution allows neuromodulation device 9004 to provide more data for post-analysis and more detailed data mining for events during use. Alternatively, compression enables faster transmission of standard-resolution clinical data.

The clinical data log file 9012 is manipulated, analysed, and efficiently presented by a clinical data viewer (CDV) 9014 for field diagnosis by a clinician, field clinical engineer (FCE) or the like. CDV 9014 is a software application installed on the Clinical Interface (CI). In one implementation, CDV 9014 opens one Clinical Data Log file 9012 at a time. CDV 9014 is intended to be used in the field to diagnose patient issues and optimise therapy for the patient. CDV 9014 may be configured to provide the user or clinician with a summary of neuromodulation device usage, therapy output, and errors, in a simple single-view page immediately after log files are compiled upon device connection.

Clinical Data Uploader 9016 is an application that runs in the background on the CI, that uploads files generated by the CPA 9010, such as the clinical data log file 9012, to a data server. Database Loader 9022 is a service which runs on the data server and monitors the patient data folder for new files. When Clinical Data Log files are uploaded by Clinical Data Uploader 9016, database loader 9022 extracts the data from the file and loads the extracted data to Database 9024.

The data server further contains a data analysis web API 9026 which provides data for third-party analysis such as by the analysis module 9032, located remotely from the data server. The ability to obtain, store, download and analyse large amounts of neuromodulation data means that the present technology can: improve patient outcomes in difficult conditions; enable faster, more cost effective and more accurate troubleshooting and patient status; and enable the gathering of statistics across patient populations for later analysis, with a view to diagnosing aetiologies and predicting patient outcomes.

### Programming the Neuromodulation Device

An important preliminary task, once a neuromodulation device has been implanted in a patient, is to assign suitable parameter values to a therapy program. The control unit will control the stimulus source, in accordance with the therapy program, to generate stimulation that ideally provides the patient with therapeutic benefit. The task of determining the suitable parameter values is known as programming or fitting the neuromodulation device. Programming generally involves an iterative process of applying certain test stimuli via the device, recording responses of the neural tissue, and based on the recorded responses, inferring or calculating the most effective parameter values for the patient. The resulting parameter values are then formed into a therapy program that may be loaded to the device to govern subsequent therapy.

The programming process may be a costly and time-consuming process, consuming both the time of the patient and the time of one or more clinicians who may be facilitating the programming process. Accordingly, there may be an incentive to minimise the number of test stimuli to be applied and the amount of information to be recorded and analysed during the programming process in order to reduce costs. This may limit the opportunity to identify a therapy program that provides the patient with the best therapeutic benefit. Furthermore, in some situations, the programming process may be best achieved while the patient performs a variety of daily tasks.

Additionally, in some situations, the patient's physiology or medical condition may change over time. These changes can result in the therapy program that was determined during the programming process no longer providing satisfactory therapeutic benefit to the patient. Therefore, it may be desirable to re-perform a programming process to determine an alternative therapy program, which defines different parameter values, in order to continue to provide the patient with satisfactory therapeutic benefit. Accordingly, the programming process may not just comprise an initial process that is performed during setup of the neurostimulation device for a new patient. Rather, the programming process may comprise a process of ongoing refinement or adaptation of the therapy program to suit the patient's needs.

In consideration of the desire to reduce clinician involvement in the programming process, and to allow for efficient ongoing refinement of the therapy program, there is provided herein a patient-responsive programming process which allows for the determination of a preferred therapy program based on patient reported outcomes. The patient-responsive programming process may be performed by the control unit 116 during operation of the neuromodulation device.

### Stimulation Set (Stimset)

A stimset as used herein defines an SEC and a set of therapy parameters that govern the stimulation pulses delivered through the SEC to the patient. The therapy parameters defined by a stimset may comprise one or more of: a pulse width, a pulse type, a number of phases, an order of phases, a number of stimulus electrode poles (two for bipolar, three for tripolar etc.). A stimset may also define a measurement electrode configuration, a target ECAP, and a stimulus rate or frequency. A stimset may be configured to suit an electrode array 150 that is implanted within the patient and is connected to the electronics module 110 to provide therapeutic stimulation to the patient. As referred to herein, an active stimset refers to the stimset that the control unit 116 is currently using to control the stimulus source to provide neural stimuli to the patient 108.

During the programming process, and at other times, the control unit may determine that it is desirable to switch from applying the active stimset to applying another stimset, referred to herein as the preferred stimset. The preferred stimset may be selected by the control unit with the aim of providing improved therapeutic benefit to the patient.

Set of Candidate Stimsets

In one embodiment, the control unit 116 is configured to determine a preferred stimset from a plurality of candidate stimsets, wherein each of the candidate stimsets define a different combination of therapy parameter values.

Candidate stimsets may be logically grouped together into one or more sets of candidate stimsets. Candidate stimsets, especially within a set of candidate stimsets, may be substantially similar to one another, differing only with regard to one or more parameters. For example, two candidate stimsets may differ only with regard to the value of the parameter defining the stimulus electrode configuration selected from the array of stimulus electrodes on the electrode array. In another example, two candidate stimsets may differ only with regard to the parameter defining the pulse width.

In one embodiment, a set of candidate stimsets may comprise candidate stimsets that represent the range of a particular therapy parameter. For example, a set of candidate stimsets may comprise a stimset for each of the pulse widths supported by the electronics module 110. In another example, a set of candidate stimsets may comprise a stimset for each stimulus electrode configuration that may be selected from the array of stimulus electrodes on the electrode array.

In one embodiment, the candidate stimsets are defined in the memory 118. The external computing device 192 may configure the memory 118 with one or more candidate stimsets. In one embodiment, the control unit 116 defines the candidate stimsets during a pre-configuration stage. In one embodiment, the control unit creates new candidate stimsets during operation. In one embodiment, the control unit modifies candidate stimsets during operation.

### Patient Reported Outcomes

A patient reported outcome (PRO) comprises an indication of the patient's satisfaction or dissatisfaction with the therapeutic benefit being provided by the current stimset. A PRO that indicates a patient's dissatisfaction with the therapeutic benefit being provided by the current stimset is an adverse PRO. A patient may be dissatisfied with a stimset if the patient 108 is experiencing pain or discomfort during stimulation in accordance with the stimset, or if the patient 108 is not experiencing a satisfactory level of therapeutic benefit

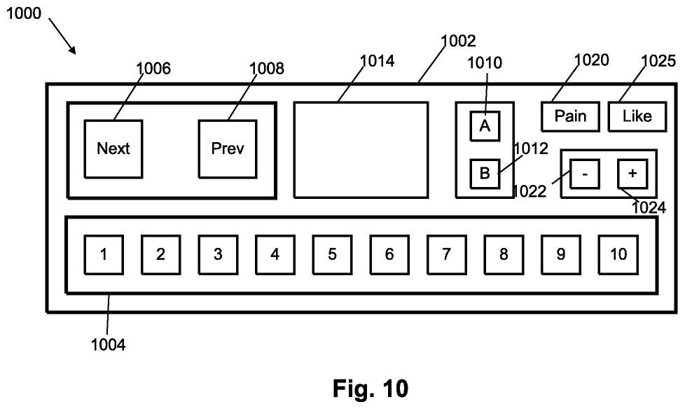

### FIG. 10—Patient Control Interface

In one embodiment, the external computing device 192 includes a patient control interface configured to transmit patient reported outcomes to the electronics module 110 via the communications channel 190. FIG. 10 illustrates a patient control interface 1000, according to one implementation of the present technology.

The communication interface 114 is configured to receive information, via communication channel 190, from the patient control interface 1000. The information may comprise a patient reported outcome, which may comprise an indication of the patient's satisfaction with the neural stimulation being provided by the electronics module 110.

In one embodiment, the patient control interface 1000 comprises a physical device. The physical device may be portable by the patient, and may be handheld by the patient. In another embodiment, the patient control interface 1000 comprises an application executing on a device that is accessible to the patient. For example, the patient control interface may comprise an application executing on the patient's mobile communication device.

In one embodiment, the patient control interface 1000 comprises a patient input interface (PII) 1002. The PII 1002 may comprise a graphical user interface (GUI) or a physical user interface. The PII 1002 comprises a plurality of input controls which may each be activated by the patient 108. The input buttons enable the patient 108 to signal to the electronics module 110 an indication of satisfaction (or dissatisfaction) with a stimset that the electronics module 110 is applying to the patient.

### Quantitative Satisfaction Indication

The PII 1002 may be used by the patient 108 to provide a quantitative indication of satisfaction with the current stimset. For example, in the embodiment of the PII illustrated in FIG. 10, the patient 108 may provide a quantitative satisfaction indication via a visual analogue scale (VAS) pain intensity score. The PII 1002 includes a plurality of (VAS) pain intensity buttons 1004 which may be used by the patient 108 to indicate an intensity of pain experienced by the patient 108. The VAS pain intensity buttons 1004 comprise ten buttons which represent an increasing intensity of pain experienced, wherein button 1 indicates minimal or no pain, and button 10 indicates an excruciating intensity of pain. The VAS pain intensity buttons 1004 may depict visual or numerical indications of pain intensity. In some embodiments, activation of the VAS pain intensity buttons 1004 above a threshold of satisfaction, e.g. five, are examples of adverse PROs.

In some embodiments, the PII may include an indication of a sliding scale which the patient 108 may use to indicate an intensity of pain experienced. In some embodiments, the PII may include only a single pain button 1020 which the patient 108 may use to indicate that the patient is experiencing pain or discomfort. The single pain button 1020 may depict an indicium of pain such as the word “pain” as illustrated in FIG. 10 or an icon representing a medicinal capsule (a “pill”), in which case the pain button 1020 may be referred to as the “pill button”. In some embodiments, activation of the pill button 1020 is an example of an adverse PRO.

### Next and Previous Buttons

The PII 1002 further comprises a Next button 1006 and a Previous button 1008. The patient 108 may activate the Next button 1006 to indicate a request for the electronics module 110 to change the active stimset to another stimset. The patient 108 may activate the Next button 1006 if they are dissatisfied with the therapeutic benefit being provided by the active stimset. In response to receiving an indication that the Next button 1006 was activated, the control unit 116 determines a preferred stimset and switches from the active stimset to the preferred stimset such that the preferred stimset becomes the active stimset. The patient may activate the Previous button 1008 if they feel that they were more satisfied with the previous stimset being applied, compared to the current stimset. In some embodiments, activation of the Previous button 1008 or the Next button 1006 are examples of adverse PROs.

### Prompt Signal

The patient control interface 1000 further comprises a screen 1014 upon which the PII can render a prompt signal. The prompt signal is configured to prompt the patient 108 to make a selection on the PII to indicate a level of satisfaction with the current stimset. The prompt signal may comprise a visual signal such as a graphic or a light, or an audible signal such as a beep, or a physical signal, such as a vibration.

### Option A or Option B

In one embodiment, the control unit 116 may be configured to provide stimulation in accordance with a first stimset (stimset A) for a period of time, then provide stimulation in accordance with a second stimset (stimset B) for a subsequent period of time. The control unit 116 may then cause the PII to prompt the patient 108 to either select a preference for stimset A, by selecting Option A button 1010, or to select a preference for stimset B, by selecting Option B button 1012. The control unit may be configured to factor in the selection of either Option A or Option B in the determination of a preferred stimset.

### Adjust Target ECAP

The PII 1002 further comprises a means to allow the patient 108 to adjust the target ECAP, which may be thought of as changing to a new stimset with a different value of the target ECAP parameter. In response to the patient 108 pressing button 1022, the patient control interface 1000 communicates an indication to the target ECAP controller 304 of the control unit 116, via communication interface 114, to decrease the target ECAP amplitude. In response to the patient 108 pressing button 1024, the patient control interface 1000 communicates an indication to the target ECAP controller 304 of the control unit 116, via communication interface 114, to increase the target ECAP amplitude. The activation of button 1022 or 1024 may comprise a patient reported outcome.

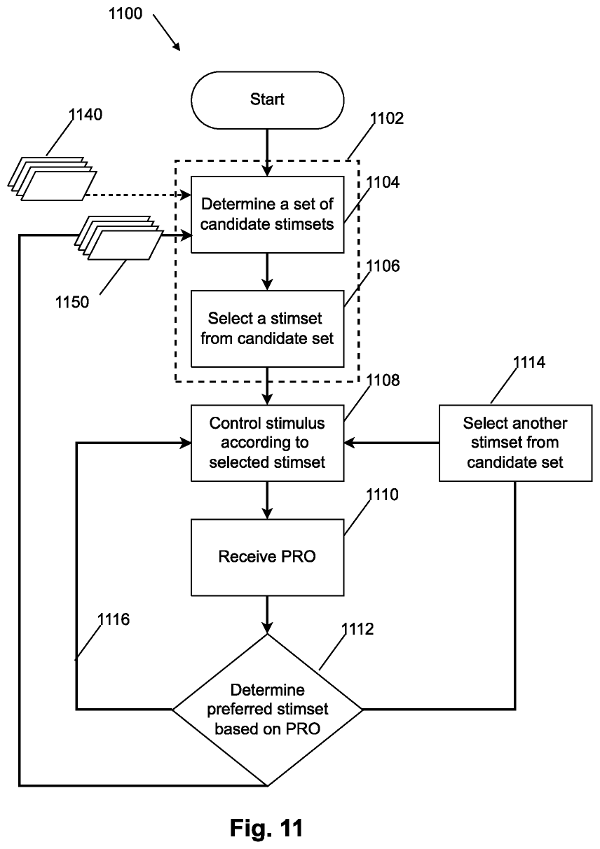

### FIG. 11—Method of Control Unit

FIG. 11 illustrates a method 1100, performed by the control unit 116, to determine a preferred stimset based on a patient reported outcome, according to one implementation of the present technology. Method 1100 comprises a configuration step 1102, comprising steps 1104 and 1106. This configuration step 1102 may not be performed by the control unit in all embodiments. In step 1104, the control unit determines a set of candidate stimsets. The control unit may select the set of candidate stimsets from a plurality of sets of candidate stimsets 1140 defined in memory 118. Alternatively, the control unit may receive an indication of the set of candidate stimsets from the external computing device 192 via the communication interface 114. In step 1106, the control unit 116 selects a first stimset from the set of candidate stimsets.

In some embodiments, the control unit determines the first stimset by determining a set of parameters for the first stimset, rather than performing steps 1104 and 1106.

In step 1108, the control unit 116 controls the stimulus source to provide the neural stimulus according to the first stimset. The first stimset is referred to as the active stimset while stimulation is being applied to the patient in accordance with the therapy parameters of the first stimset.

In step 1110, the control unit 116 receives at least one patient reported outcome (PRO). In one embodiment, the control unit receives the PRO from the patient control interface 1000 via the communication interface 114. In some embodiments, for example as described in relation to FIG. 15, the control unit 116 causes the patient control interface 1000 to prompt the patient to report a patient reported outcome.

In step 1112, the control unit 116 considers the one or more PROs received during application of the first stimset, and determines a preferred stimset based on the one or more PROs.

The consideration process that the control unit 116 performs at step 1112 to determine a preferred stimset based on the patient reported outcome may depend upon the type of PRO received by the control unit and the various programming methods performed by the control unit 116.

In one embodiment, the PRO comprises an indication that the Next button 1006 was activated. Accordingly, the control unit selects 1114 the next stimset in the set of candidate stimsets as the preferred stimset. The control unit then proceeds to step 1108.

In one embodiment, the PRO comprises an indication that the Previous button 1008 was activated. Accordingly, the control unit selects 1114 the previous stimset in the set of candidate stimsets as the preferred stimset. The control unit then proceeds to step 1108.

In one embodiment, the PRO comprises an indication that the pill button 1020 was activated. Accordingly, the control unit selects 1114 the next stimset in the set of candidate stimsets as the preferred stimset. The control unit then proceeds to step 1108.

In one embodiment, the PRO comprises an indication of pain intensity triggered by the activation of buttons 1004. In particular, the PRO comprises an indication of pain intensity level 1. The control unit considers that a pain intensity of level 1 is within acceptable levels. Accordingly, the control unit retains the first stimset as the preferred stimset and returns to step 1108. Alternatively, if the PRO comprises an indication of pain intensity (such as level 10) that is not within acceptable levels, the control unit selects 1114 the next stimset in the set of candidate stimsets as the preferred stimset and returns to step 1108.

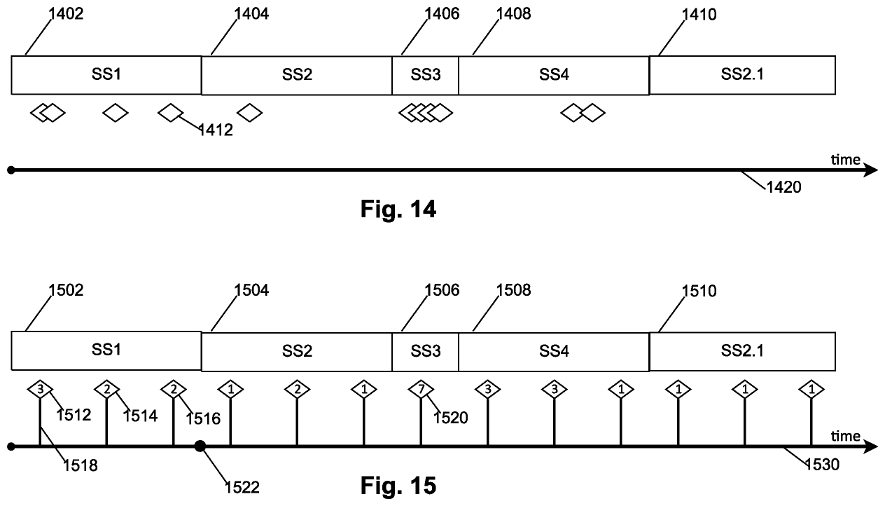

In one embodiment, for example as described in relation to FIG. 14, the control unit is configured to apply the first stimset for a set time period (e.g. 30 minutes). Accordingly, in response to the control unit receiving a PRO in step 1110 before the set time period has elapsed, the control unit records information regarding the PRO in memory, and retains the first stimset as the preferred stimset and returns to step 1108.

In one embodiment, the PRO comprises an indication to increase the target ECAP, triggered by the activation of button 1024. Accordingly, the control unit in step 1112 determines a preferred stimset to be the first stimset, with the target ECAP parameter increased, and returns to step 1108.

In one embodiment, the PRO comprises an indication to decrease the target ECAP, triggered by the activation of button 1022. Accordingly, the control unit in step 1112 determines a preferred stimset to be the first stimset, with the target ECAP parameter decreased, and returns to step 1108.

In one embodiment, in response to the control unit receiving an adverse PRO or timing out for each of the stimsets in the set of candidate stimsets, the control unit returns to step 1104 to determine a second set of candidate stimsets 1150 from the plurality 1140 of sets of candidate stimsets. In one such example, each stimset in the original set of candidate stimsets defined the same stimulus electrode configuration. In such an example, each stimset in the second set of candidate stimsets 1150 defines a stimulus electrode configuration that differs from the stimulus electrode configuration defined in the original set of candidate stimsets.

After step 1112, if the control unit 116 returns to step 1108, the active stimset is set to the preferred stimset as determined in step 1112. Accordingly, the control unit controls the stimulus source to provide the neural stimulus according to the preferred stimset.

While performing step 1108, the control unit may receive a further PRO. In response to receiving a further PRO, the control unit 116 may again transition through steps 1110 and 1112, where the control unit considers the PROs and determines another preferred stimset.

Accordingly, the control unit 116 may iterate through steps 1108, 1110 and 1112 multiple times, responding to received PROs by adjusting the stimset with the aim of providing improved therapeutic benefit to the patient.

If no further set of candidate stimsets can be defined, the method 1100 may halt after step 1112 instead of returning to step 1104. The preferred stimset from the final candidate stimset may be taken as an optimal stimset to provide improved therapeutic benefit to the patient.

### Determining a Preferred Stimset

The control unit may utilise one or more methods to determine a preferred stimset from a set of candidate stimsets. In one embodiment, the candidate stimsets are logically arranged in a tree structure, such that the candidate stimsets may be logically traversed through a tree searching methodology based on the values of the parameters defining the candidate stimsets.

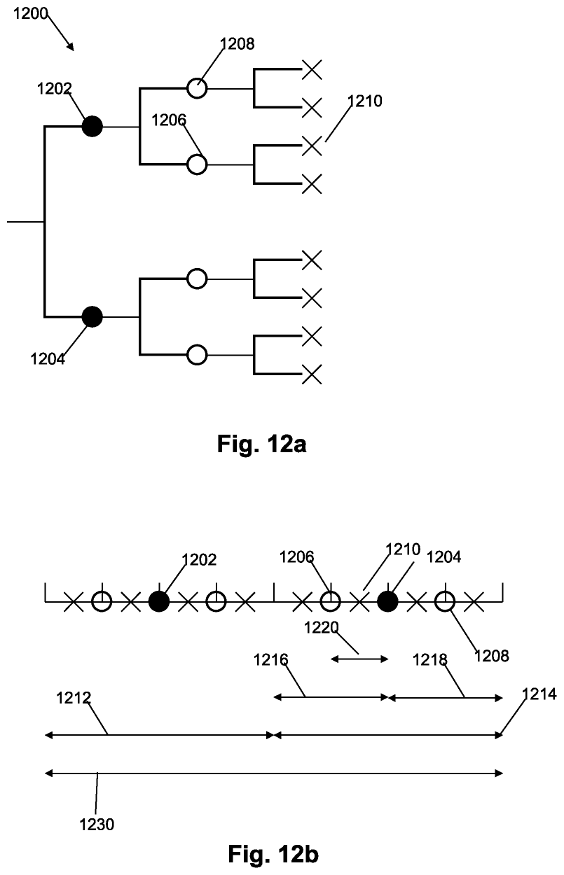

FIG. 12a illustrates an example 1200 of a binary tree-structured set of candidate stimsets. Each marker in the set 1200, e.g. the markers 1202, 1204, 1206, and 1208, represent candidate stimsets. Each candidate stimset except the “leaf” candidate stimsets marked with “X” in FIG. 12a has two child candidate stimsets. For example, the candidate stimsets 1206 and 1208 are children of the candidate stimset 1202. The “leaf” candidate stimset 1210 is one of the two children of the candidate stimset 1206.

The set 1200 is tree-structured in order to enable a binary search over one or more ranges of respective numeric parameters in the therapy parameters making up a stimset, to arrive at the optimal values of the respective parameters. In one example, illustrated in FIG. 12b, a single parameter has a range 1230 of values. All the candidate stimsets in the set 1200 have identical therapy parameter values for all parameters except the single parameter that may vary over the range 1230. The range 1230 may be partitioned into the two intervals 1212 and 1214. The candidate stimsets 1202 and 1204 are reproduced at the midpoints of the respective intervals 1212 and 1214 to indicate that their values for the variable parameter are equal to the respective midpoints of the intervals 1212 and 1214. Likewise the interval 1214 may be partitioned into the two intervals 1216 and 1218. The candidate stimsets 1206 and 1208 are reproduced at the midpoints of the respective intervals 1216 and 1218 to indicate that their values for the variable parameter are equal to the respective midpoints of the intervals 1216 and 1218. The “leaf” candidate stimset 1210 is reproduced at the midpoint of the interval 1220 to indicate that its value for the variable parameter is equal to the midpoint of the interval 1220, which is the upper partition of the interval 1216 represented by the candidate stimset 1206. The tree-structured set 1200 may have leaf candidate stimsets representing the desired resolution (final interval size) of the variable parameter.

To conduct a binary search through the set 1200, the candidate stimsets 1202 and 1204 are first compared using one of the PRO-derived metrics described below, to determine the preferred candidate stimset. The search continues by comparing the two children of the preferred candidate stimset until a leaf candidate stimset is determined to be the preferred candidate stimset. For example, if the candidate stimset 1202 were determined to be preferred after the first binary comparison, the second comparison would be between candidate stimsets 1206 and 1208. If the binary search were to end at the leaf candidate stimset 1210, this indicates that the optimal value of the variable therapy parameter (to the desired resolution) lies within the interval 1220.

Tree-structured sets of candidate stimsets may be implemented with higher numbers of children for each non-leaf candidate stimset, meaning the choice at each stage is no longer binary, but ternary (among three children) or quaternary (among four children), etc. Increasing the number of children decreases the number of comparisons required to reach optimal value of the variable parameter to the desired resolution, at the cost of greater complexity in each comparison.

If there are multiple variable therapy parameters to be optimised, the candidate stimsets' values of the variable parameters may be chosen such that comparisons at each level of the tree cycle through the variable therapy parameters. In an example, the choice between candidate stimsets 1202 and 1204 may represent a halving of the initial range of variation of the value of a first variable parameter, while the subsequent choice between candidate stimsets 1206 and 1208 may represent a halving of the initial range of variation of the value of a second variable parameter, and so on.

### Example Scenarios

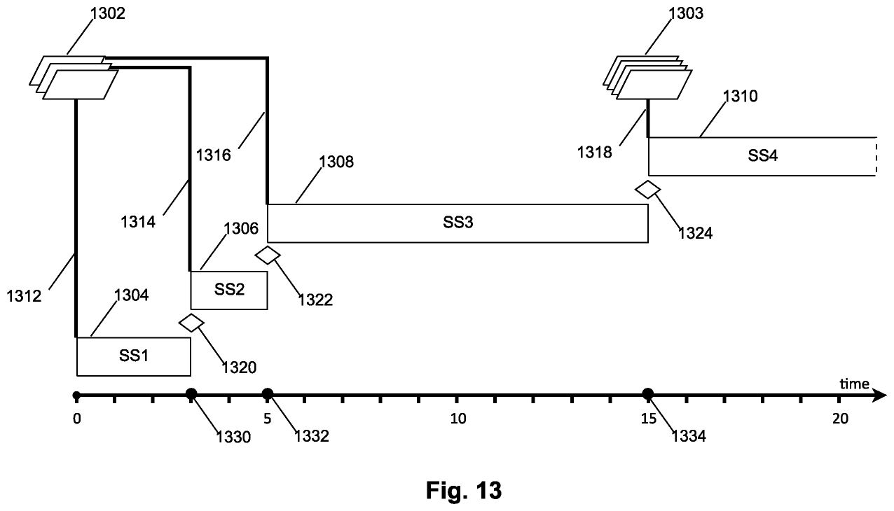

FIGS. 13 to 15 each illustrate the operation of the control unit 116 during an example scenario and an example mode of operation of the control unit. The control unit may apply one or more of the concepts depicted in FIGS. 13 to 15, and described herein, in combination or at different times during operation of the control unit.

### FIG. 13—Determining Preference Based on Time

During a programming process, the control unit 116 may be configured to trial a plurality of different stimsets of a set of candidate stimsets to determine a preferred stimset. In one embodiment, the control unit 116 is configured to determine a preferred stimset based on a measure of time that the control unit controls the stimulus source in accordance with a stimset before the control unit receives an adverse PRO from the communication interface.

A relatively long time period for which the control unit applies the stimset before the control unit receives an adverse PRO may be indicative of the patient's satisfaction with the stimset. Conversely, if the control unit receives an adverse PRO after applying a stimset for only a short period of time, this may be indicative of the patient's dissatisfaction with the stimset.

FIG. 13 illustrates an example scenario in which the control unit 116 applies a time-based method to determine a preferred stimset from a plurality of sets of candidate stimsets, according to one implementation of the present technology.

The control unit 116 is configured with a first set of three candidate stimsets SS1, SS2 and SS3 (collectively 1302). Each of the three candidate stimsets 1302 define the application of neural stimuli in accordance with different parameters. For example, the three candidate stimsets 1302 may differ with regard to the stimulus electrode configuration, such that stimulus is generated at a different position on the electrode array 150 and therefore a different region of the patient's spinal cord is stimulated by the neural stimuli. In another example, the three candidate stimsets 1302 may differ with regard to pulse width such that each of the three candidate stimsets provides a different stimulus sensation for the patient.

The control unit 116 selects 1312 a first stimset SS1 from the first set of candidate stimsets 1302. The control unit controls the stimulus source in accordance with the SS1 stimset for time period 1304. At time 1330, the control unit receives an adverse PRO 1320 via the communication interface 114. The adverse PRO 1320 indicates that the patient 108 is dissatisfied with the therapeutic benefit of the first stimset SS1. For example, the adverse PRO 1320 comprises an indication that the patient has pressed the pill button 1020.

In response to receiving adverse PRO 1320, the control unit 116 selects a second stimset SS2 from the first set of candidate stimsets 1302. The control unit controls the stimulus source in accordance with the SS2 stimset for time period 1306. At time 1332, the control unit receives an adverse PRO 1322 via the communication interface. The adverse PRO 1322 indicates that the patient 108 is dissatisfied with the therapeutic benefit of the second stimset SS2. For example, the adverse PRO 1322 comprises an indication that the patient has pressed the pill button 1020.

In response to receiving adverse PRO 1322, the control unit 116 selects the third stimset SS3 from the first set of candidate stimsets 1302. The control unit controls the stimulus source in accordance with the SS3 stimset for time period 1308. At time 1334, the control unit receives an adverse PRO 1324 via the communication interface. For example, the adverse PRO 1324 comprises an indication that the patient has pressed the pill button 1020.

After applying each of the candidate stimsets from the set of candidate stimsets 1302, the control unit 116 determines a second set of candidate stimsets based on the time periods 1304, 1306, and 1308 that the control unit applied the candidate stimsets SS1, SS2 and SS3 respectively, before an adverse PRO was received.

In an example, stimset SS1 was applied for 3 minutes before time 1330. Stimset SS2 was applied for 2 minutes for time 1332. Stimset SS3 was applied for 10 minutes before time 1334, which may indicate that the patient 108 considered that the stimset SS3 provided more satisfactory therapeutic benefit compared to SS1 or SS2.

In response to the time periods for which each stimset was applied before the control unit received an adverse PRO, the control unit selects a second set of candidate stimsets 1303 which have therapy parameter values which are similar to at least one of the therapy parameter values of SS3.

In an example, the control unit uses the Bayesian methodology described below to select the second set of candidate stimsets 1303. The time-based method illustrated in FIG. 13 and the Bayesian methodology may be used repeatedly until a stimset is found that gives the most satisfactory therapeutic benefit.

In another example, in which the candidate stimsets SS1 to SS3 are the first level of a ternary tree-structured set of candidate stimsets as described above, the control unit selects the second set 1303 of candidate stimsets as the children of the preferred candidate stimset SS3.

At time 1334, the control unit 116 selects a candidate stimset SS4 from the second set of candidate stimsets 1303, and controls the stimulus source in accordance with the SS4 stimset.

In the example illustrated in FIG. 13, the adverse PROs received by the control unit 116 comprises an indication that the patient has pressed the pill button 1020. In some embodiments, the adverse PRO may also comprise information regarding the intensity of the pain or discomfort experienced by the patient 108, the location of pain or discomfort experienced by the patient, or another indication of the dissatisfaction of the patient with the therapeutic benefit of the stimulus.

FIG. 14—Counting Adverse PROs

It may be beneficial to determine a preferred stimset by applying each of a selection of candidate stimsets in turn, and considering the patient reported outcomes that are generated by the patient during the application of each of the candidate stimsets.

FIG. 14 illustrates an example in which the control unit 116 is configured to determine a preferred stimset based on a consideration of the number of adverse PROs that are received by the control unit during the application of candidate stimsets, according to one implementation of the present technology. In particular, the control unit is configured to determine a preferred stimset, based on the adverse PROs received during application of each of a set of four candidate stimsets SS1 to SS4. In the example illustrated in FIG. 14, adverse PROs are illustrated by diamond shapes, such as PRO 1412, occurring at times along timespan 1420. The adverse PROs may comprise an indication that the patient 108 has pressed the pill button 1020.

The control unit 114 is configured to control the stimulus source in accordance with each candidate stimset for a set period of time (e.g. 30 minutes), before switching to the next candidate stimset. However, in the event that the receipt of PROs indicate that the active stimset is causing the patient unacceptable pain or discomfort, the control unit 116 is configured to switch to the next candidate stimset before the set period of time has elapsed.

During the time period 1402, in which the control unit 116 controls the stimulus source in accordance with candidate stimset SS1, the control unit receives 4 adverse PROs via the communication interface 114. During the time period 1404, in which the control unit 116 controls the stimulus source in accordance with candidate stimset SS2, the control unit receives 1 adverse PRO.

During the time period 1406, in which the control unit 116 controls the stimulus source in accordance with candidate stimset SS3, the control unit receives 4 adverse PROs in quick succession. This quick succession of adverse PROs indicates that SS3 causing the patient an unacceptable level of pain or discomfort. Accordingly, the control unit 116 switches to controlling the stimulus source in accordance with the fourth candidate stimset SS4.

During the time period 1408, in which the control unit 116 controls the stimulus source in accordance with candidate stimset SS4, the control unit receives 2 adverse PROs.

After applying each candidate stimset of the set of four candidate stimsets SS1 to SS4, the control unit 116 determines that, based on the number of adverse PROs received, candidate stimset SS2 provides the patient with the most satisfactory therapeutic benefit. Accordingly, the control unit selects a second set of candidate stimsets which have therapy parameter values that are similar to those of candidate stimset SS2. The first of these candidate stimsets, illustrated as SS2.1 in FIG. 14, is applied during time period 1410.

In an example, the control unit uses the Bayesian methodology described below to select the second set of candidate stimsets. The count-based method illustrated in FIG. 14 and the Bayesian methodology may be used repeatedly until a stimset is found that gives the most satisfactory therapeutic benefit.

In another example, in which the candidate stimsets SS1 to SS4 are the first level of a quaternary tree-structured set of candidate stimsets as described above, the control unit selects the second set of candidate stimsets as the children of the preferred candidate stimset SS2.

### FIG. 15—Prompting Patient to Provide PROs

It may be beneficial to determine a preferred stimset by prompting the patient 108 to provide patient reported outcomes to indicate the patient's satisfaction with an applied candidate stimset.

In one embodiment the control unit 116 is configured to cause the patient control interface 1000 to generate a prompt signal via the PII 1002 to prompt the patient 108 to input an indication of satisfaction with the active stimset. In one embodiment, the control unit 116 transmits a signal to the patient control interface 1000 via the communication interface 114, which causes the patient control interface 1000 to generate the prompt signal. The prompt signal may comprise a visual signal depicted in the screen 1014. Alternatively, the prompt signal may comprise an audible signal or a physical signal such as a vibration.

FIG. 15 illustrates an example scenario in which the control unit 116 utilises prompts to obtain PROs, which the control unit uses to determine a preferred stimset, according to one implementation of the present technology. FIG. 15 illustrates the occurrence of events over a time span indicated by timeline 1530. FIG. 15 illustrates plurality of time periods in which the control unit 116 controls the stimulus source in accordance with a sequence of candidate stimsets.

The time at which the control unit 116 causes the patient control interface 1000 to generate a prompt signal is represented by vertical lines (such as 1518) extending from timeline 1530 and terminating in the PRO (such as 1512) that was triggered by the generation of the prompt signal. Each of the PROs illustrated in FIG. 15 indicates a pain intensity score, as selected by the patient using the buttons 1004, wherein the scores are selected from 1 to 10, with 1 indicating mild or no pain intensity, and 10 indicating the highest level of pain intensity.

In the example illustrated in FIG. 15, the control unit 116 is configured to apply each of four candidate stimsets for a set period of time (e.g. 30 minutes), then transition to applying the next candidate stimset for the next set period of time. During these set periods of time, the control unit 116 triggers prompt signals and receives PROs as provided by the patient.

During the time period 1502, in which the control unit 116 controls the stimulus source in accordance with candidate stimset SS1, the control unit triggered a prompt signal 3 times, resulting in the receipt by the control unit of PROs 1512, 1514 and 1516, indicating pain intensity levels of 3, 2 and 2, respectively. During the time period 1504, in which the control unit 116 controls the stimulus source in accordance with candidate stimset SS2, the control unit receives three PROs indicating pain intensity levels of 1, 2, and 1, respectively.

During the time period 1506, in which the control unit 116 controls the stimulus source in accordance with candidate stimset SS3, the control unit receives a PRO which indicates a pain intensity of 7. As a pain intensity of 7 indicates that SS3 is not providing satisfactory therapeutic benefit to the patient (an adverse PRO), the control unit promptly transitions to the next candidate stimset SS4.

During the time period 1508, in which the control unit 116 controls the stimulus source in accordance with candidate stimset SS4, the control unit receives three PROs indicating pain intensity levels of 3, 3, and 1, respectively.

The control unit 116 considers the received PROs, including the pain intensities indicated by the PROs, to determine which of the four candidate stimsets SS1-4 provided the best performance in terms of therapeutic benefit to the patient. Considering only pain intensity in this example, the control unit 116 determines that candidate stimset SS2 provided the best therapeutic performance. Accordingly, the control unit determines a second set of candidate stimsets which have parameters similar to the parameters of candidate stimset SS2. The first of these candidate stimsets, illustrated as SS2.1 in FIG. 15, is applied during time period 1510.

In an example, the control unit uses the Bayesian methodology described below to select the second set of candidate stimsets. The pain-intensity-score-based method illustrated in FIG. 15 and the Bayesian methodology may be used repeatedly until a stimset is found that gives the most satisfactory therapeutic benefit.

In another example, in which the candidate stimsets SS1 to SS4 are the first level of a quaternary tree-structured set of candidate stimsets as described above, the control unit selects the second set of candidate stimsets as the children of the preferred candidate stimset SS2.

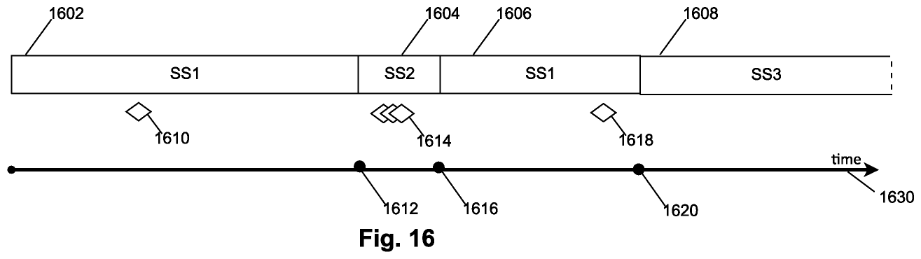

### FIG. 16—Changing Stimset to Determine Patient's Response

In some situations, a suitable, but not ideal stimset may have been determined for a patient. For example, the stimset may provide the patient with satisfactory therapeutic benefit for most of the time, and in most situations, but there may be situations in which the patient experiences discomfort or pain during application of the stimset. Accordingly, there may be an alternative stimset which would provide the patient with more consistent or thorough therapeutic benefit.

In one embodiment the control unit 116 is configured to occasionally switch from applying a previously determined default stimset to applying a candidate stimset. The control unit is configured to determine the suitability of the candidate stimset, compared to the default stimset based on whether the control unit receives PROs during application of the candidate stimset, and the nature of the received PROs, if any.

FIG. 16 illustrates an example scenario in which the control unit 116 switches from applying a default stimset to applying a candidate stimset, according to one implementation of the present technology. FIG. 16 illustrates the occurrence of events over a time span indicated by timeline 1630. FIG. 16 may not be drawn to scale and timeline 1630 may not illustrate a linear progression of time.

Stimset SS1 is the default stimset that the control unit 116 has previously determined is suitable for providing at least some therapeutic benefit for the patient.

During the time period 1602, in which the control unit 116 controls the stimulus source in accordance with default stimset SS1, the control unit receives a single adverse PRO 1610. At time 1612, the control unit 116 switches to controlling the stimulus source in accordance with candidate stimset SS2. During the time period 1604, the control unit receives three adverse PROs 1614 in quick succession. In response to receiving the three PROs 1614, the control unit determines that candidate stimset SS2 does not provide an improved therapeutic benefit compared to default stimset SS1. At time 1616, the control unit reverts to applying default stimset SS1. The control unit receives a single adverse PRO 1618 during the time period 1606.

At time 1620, the control unit 116 switches to controlling the stimulus source in accordance with candidate stimset SS3. During the time period 1608, the control unit does not receive any PROs. In response to receiving no PROs, the control unit determines that candidate stimset SS3 may provide an improved therapeutic benefit compared to default stimset SS1. Accordingly, the control unit continues to control the stimulus source in accordance with candidate stimset SS3.

### System Data

In one embodiment, the control unit 116 is configured to factor in system data when determining a preferred stimset, in step 1112. System data may pertain to the operation of the electronics module 110. System data may comprise an indication of power consumption during the application of a candidate stimset. Accordingly, patient preference may be balanced with power consumption in converging on the optimal combination of parameters or stimsets.

### Multidimensional Parameter Space

A stimset may be comprised of a plurality of parameter values, wherein each parameter value is associated with a therapy parameter (such as pulse width, pulse type, stimulus electrode configuration etc.). Accordingly, all the possible combinations of parameter values that may define a stimset may be considered to make up a multidimensional parameter space, or multidimensional matrix of parameter values. Similarly, a set of predefined candidate stimsets (e.g. as stored in memory 118) with different combinations of parameter values may also be considered to populate a multidimensional parameter space, or multidimensional matrix of parameter values. Accordingly, the process of determining a preferred stimset may comprise a search through the multidimensional parameter space.

In one embodiment, the control unit 116 is configured to determine a preferred stimset by performing an iterative search through a tree-structured set of candidate stimsets representing a multidimensional parameter space of parameter values, as described above. The control unit may perform the search based on a consideration of the PROs received by the control unit during application of one or more stimsets in the set of candidate stimsets. For example, if multiple adverse PROs were received during stimulation in accordance with a stimset defining a stimulus electrode configuration of stimulus electrodes at the distal end of the electrode array, when determining a preferred stimset, the control unit may disregard all candidate stimsets that define stimulus electrode configurations with stimulus electrodes at the distal end of the electrode array.

In one embodiment, the control unit is configured to apply rules mapping preferences to determine a preferred stimset among multiple candidate stimsets.

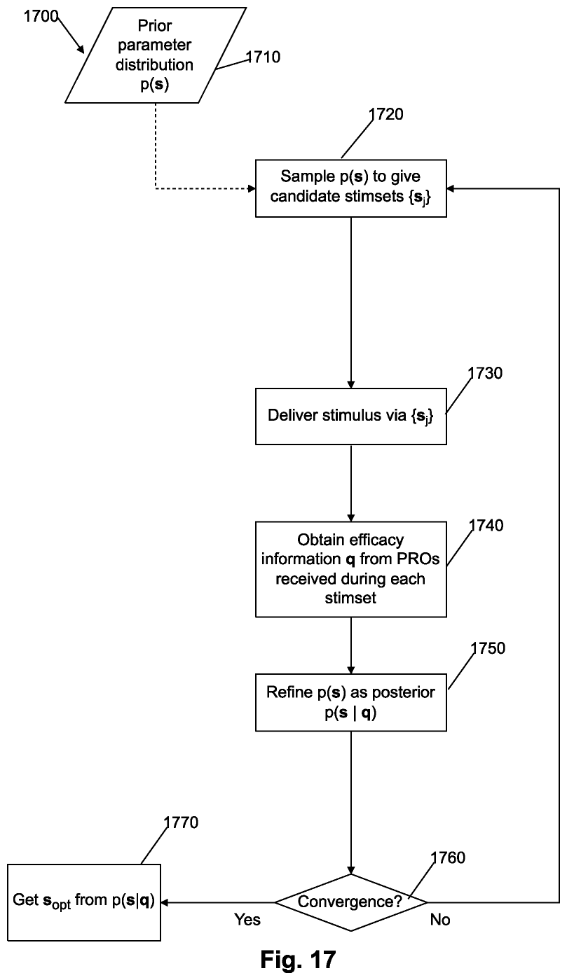

### FIG. 17—Bayesian Methodology

In one embodiment, the control unit is configured to apply Bayesian methods to determine a preferred stimset. FIG. 17 is a flow chart illustrating a method 1700 for determining a preferred stimset or a preferred set of candidate stimsets from a set of candidate stimsets, according to one implementation of the present technology. In one embodiment, method 1700 is performed by control unit 116.

A preferred stimset or a preferred set of candidate stimsets may be determined through the consideration of one or more of: PROs received by the control unit in response to application of a candidate stimset; PROs received by the control unit in response to application of other candidate stimsets; attributes of the evoked responses as measured by measurement electrode configurations; or measurement parameters sampled from prior distributions of the candidate stimsets.

Method 1700 defines a process in which prior distributions p(s) 1710 of candidate stimsets are iteratively refined using efficacy information q derived from patient reported outcomes (PROs), until a stopping criterion is reached, upon which the refined distributions may be used to determine a preferred set of candidate stimsets, or a preferred stimset.

The method 1700 starts at step 1720. In step 1720, the control unit 116 samples a prior distribution p(s) 1710 of candidate stimsets to obtain a sample candidate stimset {sⱼ}. In one implementation of step 1720, the distribution p(s) 1710 of candidate stimsets is defined during a prior execution of method 1700. In other implementations of step 1720, the distribution p(s) 1710 of candidate stimsets comprises a default distribution.

In step 1730, the control unit 116 controls the pulse generator 124 to generate stimuli in accordance with the sample candidate stimset {sⱼ}.

In step 1740, the control unit 116 obtains one or more efficacy measures q of the stimulus provided by the electronics module 110 in accordance with the stimset {sⱼ} delivered at step 1730. The one or more efficacy measures q may be determined based on one or more PROs determined by the control unit 116 in response to step 1730. In some embodiments, the efficacy measures q may further comprise one or more quality measures. International Patent Publication no. WO2021007615 by the present applicant, the contents of which are herein incorporated by reference, describes a method of obtaining a quality measure (the Signal Quality Indicator or SQI) from a set of (stimulus intensity, response intensity) pairs. Alternatively, Australian Provisional Patent Application no. 2021904237 by the present applicant describes a method of obtaining a quality measure (the Growth Curve Quality Index or GCQI) from a set of (stimulus intensity, response intensity) pairs. Alternatively, the quality measure may be derived from PROs received by the control unit 116. The one or more quality measures may be assembled in step 1740 into a quality vector.

In step 1750, the control unit 116 refines the distribution p(s) using the evidence of the efficacy measures q. In one implementation, Bayes' rule for refining a prior distribution into a posterior distribution given some evidence q may be used at step 1750. Bayes' rule states that the prior distribution p(s) may be refined into the posterior distribution p(s|q) as follows:

$$p ( s \vert q ) = \frac{p ( q \vert s ) \cdot p ( s )}{p ( q )} \qquad ( 3 )$$

where p(q|s) is the likelihood of obtaining the efficacy quality vector q given the stimset s, and p(q) is the distribution of the efficacy vector q.

In step 1790, the control unit 116 tests whether the refined, posterior distributions p(s|q) has converged sufficiently that the iteration may be ended. Convergence may be assessed by comparison of the prior distribution p(s) with the posterior distribution p(s|q).

If not converged (“No”), the method 1700 returns to step 1720. On this and subsequent iterations of step 1720, the original prior distribution 1710 has been replaced by the posterior distribution p(s|q) from the preceding iteration of step 1750.

If converged (“Yes”), step 1760 obtains the most suitable stimset sₒₚₜ from the converged posterior distribution p(s|q). In one implementation of step 1760, the modes (peak locations) of the posterior distributions are obtained as the most suitable stimset sₒₚₜ.

In other implementations of step 1760, other stopping criteria may be used, such as a fixed number of iterations being reached, or the standard deviation relative to the mean (coefficient of variation) along one or more of the component axes of the samples so is below some threshold. Alternatively, if after a certain number of iterations it is clear no convergence is occurring in the one or both of the distributions, the control unit 116 may halt method 1700 and indicate that no suitable stimset can be found.

The method 1700 may be performed by the control unit 116 during operation of the electronics module 110. Additional executions of the method 1700 may take place out of clinic, at therapy time, either periodically on a schedule, or triggered by an event, such as the receipt of a PRO, to determine whether the active stimset, defining the current measurement electrode configuration and parameters, should be changed to more suitable stimset on account of a change in circumstances such as lead migration.

In an alternative implementation, the control unit 116 may also optimise a measurement electrode configuration vector r and a measurement parameter vector m given the stimset s. This could be done by working with a joint distribution p(m, r|s), or separate distributions p(r|s) and p(m|s, r). A prior joint distribution p(m, r|s) may be derived using the stimulus program vector s and either or both of the prior patient data, prior PROs and the propagation model parameters. A method similar to the method 1700 may be used to obtain a suitable measurement electrode configuration vector rₒₚₜ and a suitable measurement parameter vector mₒₚₜ for the measurement electrode configuration rₒₚₜ, for a given stimset s by sampling and refining a joint distribution p(m, r|s), optionally starting with a prior joint distribution p(m, r|s) and ending by obtaining the most suitable measurement electrode configuration vector rₒₚₜ and measurement parameter vector mₒₚₜ from a converged posterior distribution p(m, r|s, q).

### Multi-Stimset Programs

A multi-stimset program configures the neuromodulation device to deliver stimuli from multiple stimsets at once, by interleaving their respective stimulus pulses over a single stimulus period. Multi-stimset programs are utilised to allow multiple painful areas to be treated at the same time. However, the configuration process of manually determining the optimal combination of therapy parameters in each stimset making up a multi-stimset program is time-consuming.

Instead, a configuration process may automatically set up a multi-stimset program with multiple default stimsets whose stimulus electrode configurations are widely-spaced around the electrode array and default therapy parameters for each stimset.

While executing one or more of the above-described methods to optimise the therapy parameters within each stimset, the control unit may also carry out the following procedure to refine the stimulus electrode configurations of the stimsets. One of the stimsets is chosen at random and disabled for a period, and a dissatisfaction metric based on the PROs received during the period of disablement is computed using one of the above-described methodologies. The greater the dissatisfaction metric, the higher is the preference rating of the disabled stimset. The preference ratings are used to refine the stimulus electrode configurations of the stimsets over time, for example using a Bayesian approach. The result will be a convergence to the stimulus electrode configuration for each stimset that gives the most satisfactory therapeutic benefit.

### Decreasing Target ECAP

In some circumstances, as the patient becomes accustomed to the neural stimulus provided by the electronics module 110, it may be feasible to reduce the target ECAP amplitude so as to reduce the power consumption of the implantable device whilst still providing satisfactory therapeutic benefit to the patient.

In one embodiment, the control unit 116 is configured to decrease the target ECAP, as controlled by the target ECAP controller 304, without receiving an indication from the patient to decrease the target ECAP. The control unit 116 then determines whether the decreased target ECAP is unsuitable if the control unit receives one or more adverse PROs from the patient control interface 1000.

In one embodiment, the control unit 116 is configured to decrease the target ECAP, from a set target ECAP amplitude to a decreased target ECAP amplitude, in response to the control unit 116 determining that the patient 108 is asleep. The control unit 116 may determine that the patient is asleep based on a pre-programmed sleep schedule stored in memory 118.

In response to receiving an adverse PRO, the control unit 116 is configured to revert the target ECAP to the set target ECAP amplitude. In response to the control unit 116 determining that the patient is asleep again (e.g. at the next night time), the control unit is configured to decrease the target ECAP by a smaller amount, from the set target ECAP amplitude to a decreased target ECAP amplitude that is slightly larger than the previous decreased target ECAP amplitude.

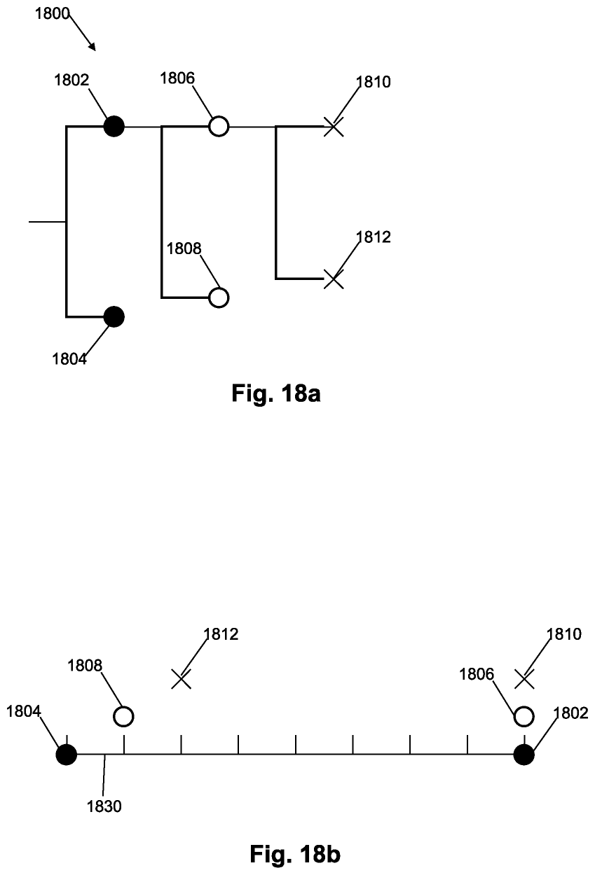

One embodiment uses a binary tree-structured set 1800 of candidate stimsets as illustrated in FIG. 18a and FIG. 18b according to one implementation of the present technology. The candidate stimsets in the tree-structured set 1800 have parameter values that are identical, except their values of the target ECAP parameter may differ. The first pair of candidate stimsets 1802 and 1804 have target ECAP values at the upper and lower ends of the range 1830 respectively. The second pair of candidate stimsets 1806 and 1808 have target ECAP values that are at the upper end and slightly above the lower end of the range 1830 respectively. The third pair of candidate stimsets 1810 and 1812 have target ECAP values that are at the upper end and slightly further above the lower end of the range 1830 respectively. Further pairs may be included in the set 1800 until the target ECAP value of the lower candidate stimset is as close as may be to the upper limit of the range 1830.

In one embodiment, the tree-structured set 1800 may be binary searched as described above in relation to FIG. 12a until a leaf candidate stimset is reached. The final leaf candidate stimset contains a “compromise” value of the target ECAP parameter that is the smallest (and therefore lowest power-consuming) value consistent with satisfactory therapeutic benefit during sleep.

A similar approach may be taken to find the compromise value of other therapy parameters with the potential to reduce power consumption, such as stimulus frequency and pulse width.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

1. An implantable device for controllably delivering a neural stimulus, the device comprising:
a plurality of electrodes including one or more stimulus electrodes and one or more measurement electrodes;
a stimulus source configured to provide neural stimuli to be delivered via the one or more stimulus electrodes to a neural pathway of a patient in order to evoke a neural response on the neural pathway;
measurement circuitry configured to capture signal windows sensed on the neural pathway via the one or more measurement electrodes subsequent to respective neural stimuli; and
a control unit configured to implement closed-loop neurostimulation therapy by:
controlling the stimulus source to provide a neural stimulus according to a stimulus intensity parameter;
measuring a characteristic of the signal window;
computing a feedback variable from an intensity of an evoked neural response in the signal window;
adjusting the stimulus intensity parameter using the feedback variable; and
repeating the controlling, measuring, computing, and adjusting so as to maintain the feedback variable at a target, thereby obtaining multiple measured intensities of neural responses,
wherein the control unit is further configured to compute one or more quantitative indicators of efficacy of the closed-loop neurostimulation therapy using the measured characteristics of the signal windows.

2. The implantable device of claim 1, wherein the characteristic of the signal window is the intensity of the evoked neural response in the signal window.

3. The implantable device of claim 2, wherein the control unit is configured to compute the one or more quantitative indicators by:
estimating a posture of the patient during an interval of a night using the intensities of the evoked neural responses and corresponding values of the stimulus intensity parameter over the interval; and
analysing a plurality of posture estimates during respective intervals of the night to compute an indicator of sleep quality for the night.

4. The implantable device of claim 2, wherein the control unit is configured to compute the one or more quantitative indicators by:
estimating a heart rate variability of the patient using the intensities of the evoked neural responses; and
computing the one or more quantitative indicators from the estimated heart rate variability.

5. The implantable device of claim 1, wherein the characteristic of the signal window is an intensity of a non-evoked neural response in the signal window.

6. The implantable device of claim 5, wherein the control unit is configured to compute the one or more quantitative indicators by:
determining an amount of non-evoked neural activity during an interval of a night using the intensities of non-evoked neural responses over the interval;
detecting REM sleep over the interval from the amount of non-evoked neural activity; and
analysing a plurality of intervals to compute an amount of REM sleep for the night, and
computing an indicator of sleep quality for the night from the amount of REM sleep for the night.

7. The implantable device of claim 1, wherein the control unit is further configured to compare the one or more quantitative indicators with respective ranges.

8. The implantable device of claim 7, wherein the control unit is further configured to transmit an indication to a user, based on the comparing.

9. The implantable device of claim 7, wherein the control unit is further configured to adjust a parameter of the closed-loop neurostimulation therapy, based on the comparing.

10. The implantable device of claim 1, wherein the control unit is further configured to adjust a parameter of the closed-loop neurostimulation therapy before computing the one or more quantitative indicators.

11. The implantable device of claim 10, wherein the control unit is further configured to compare the one or more quantitative indicators with respective ranges.

12. The implantable device of claim 11, wherein the control unit is further configured to confirm the adjustment to the parameter, based on the comparing.

13. The implantable device of claim 11, wherein the control unit is further configured to cancel the adjustment to the parameter, based on the comparing.

14. An automated method of controllably delivering a neural stimulus, the method comprising:
controlling a stimulus source to provide a neural stimulus to be delivered, via one or more stimulus electrodes, to a neural pathway of a patient in order to evoke a neural response on the neural pathway, the neural stimulus being delivered according to a stimulus intensity parameter;
capturing a signal window sensed on the neural pathway, via one or more measurement electrodes, subsequent to the neural stimulus;
measuring a characteristic of the signal window;
computing a feedback variable, from an intensity of an evoked neural response in the signal window;
adjusting the stimulus intensity parameter using the feedback variable; and
repeating the controlling, measuring, computing, and adjusting so as to maintain the feedback variable at a target, thereby obtaining multiple measured intensities of neural responses,
wherein the control unit is further configured to compute one or more quantitative indicators of efficacy of the closed-loop neurostimulation therapy using the measured characteristics of the signal window.

15. The method of claim 14, wherein the characteristic of the signal window is the intensity of the evoked neural response in the signal window.

16. The method of claim 15, wherein the computing the one or more quantitative indicators comprises:
estimating a posture of the patient during an interval of a night using the intensities of the evoked neural responses and corresponding values of the stimulus intensity parameter over the interval; and
analysing a plurality of posture estimates during respective intervals of the night to compute an indicator of sleep quality for the night.

17. The method of claim 15, wherein the computing the one or more quantitative indicators comprises:
estimating a heart rate variability of the patient using the intensities of the evoked neural responses; and
computing the one or more quantitative indicators from the estimated heart rate variability.

18. The method of claim 14, wherein the characteristic of the signal window is an intensity of a non-evoked neural response in the signal window.

19. The method of claim 18, wherein the computing the one or more quantitative indicators comprises:
determining an amount of non-evoked neural activity during an interval of a night using the intensities of non-evoked neural responses over the interval;
detecting REM sleep over the interval from the amount of non-evoked neural activity; and
analysing a plurality of intervals to compute an amount of REM sleep for the night, and
computing an indicator of sleep quality for the night from the amount of REM sleep for the night.

20. The method of claim 14, further comprising comparing the one or more quantitative indicators with respective ranges.

21. The method of claim 20, further comprising transmitting an indication to a user, based on the comparing.

22. The method of claim 20, further comprising adjusting a parameter of the closed-loop neurostimulation therapy, based on the comparing.

23. The method of claim 14, further comprising adjusting a parameter of the closed-loop neurostimulation therapy before computing the one or more quantitative indicators.

24. The method of claim 23, further comprising comparing the one or more quantitative indicators with respective ranges.

25. The method of claim 24, further comprising confirming the adjustment to the parameter, based on the comparing.

26. The method of claim 24, further comprising discarding the adjustment to the parameter, based on the comparing.
